(19) United States
(12) Patent Application Publication
(10) Pub. No.: US 2023/0181888 A1
(43) Pub. Date: Jun. 15, 2023

(54) DEVICES, METHODS, AND SYSTEMS FOR SUBCLAVIAN VEIN CATHETER PLACEMENT

(71) Applicant: SCV DELIVERY SYSTEMS LLC, WALTHAM, MA (US)

(72) Inventors: Subir RAY, WALTHAM, MA (US); KUOWEI CHANG, WALTHAM, MA (US); SANLU Y CHANG, WALTHAM, MA (US); KRISTOFER LACARRERE, WALTHAM, MA (US); Alfred SALLETTE, WALTHAM, MA (US)

(21) Appl. No.: 17/995,925

(22) PCT Filed: Apr. 7, 2021
(86) PCT No.: PCT/US2021/026116

§ 371 Date: Oct. 10, 2022

Related U.S. Application Data
(60) Provisional application No. 63/007,018, filed on Apr. 8, 2020.

(51) Int. Cl. A61M 39/02; A61M 25/06; A61M 25/00; A61M 39/04

(52) U.S. Cl. CPC A61M 39/0247; A61M 25/06; A61M 25/0097; A61M 39/045; A61M 2039/027; A61M 2039/0282; A61M 2039/0291; A61M 2039/0258

(57) ABSTRACT

A connector hub for a needle assembly, the connector hub comprising a first portion located at a first end section of the connector hub, the first portion comprising a first longitudinal axis, a second portion located at a second end section of the connector hub opposite the first end, the second portion comprising a second longitudinal axis, a medial portion located at a middle section of the connector hub between the first and second end sections, the medial portion comprising a medial longitudinal axis arranged at an angle relative to the first longitudinal axis, and arranged at an angle relative to the second longitudinal axis, and at least one insertion port located on the medial portion, wherein the insertion port comprises an insertion port longitudinal axis, wherein the insertion port longitudinal axis is arranged at an angle relative to the medial longitudinal axis.

### FIELD OF THE INVENTION

Devices, methods, and systems for safe and efficacious centerline catheter (i.e., central venous catheter) placement in a patient in need thereof are described. A specially formed needle assembly having a needle, a syringe and a specially formed connector hub connecting the needle with the syringe may be used for subclavian vein penetration via infraclavicular site for central venous access. The specially formed connector hub serves to align the syringe at an angle relative to the needle and provides an insertion port for inserting a catheter guide wire through the connector hub and needle and into the vein. The insertion port is also arranged at an angle relative to the body of the connector hub and/or to the syringe and/or to the introducer needle. The invention facilitates accurate and safe entry into subclavian vein with reduced risk of pneumothorax (i.e., a collapsed lung) and other injuries.

### BACKGROUND OF THE INVENTION

A central venous catheter (alternatively known as a centerline catheter) is a catheter placed into a large vein in the neck (i.e., the internal jugular vein), chest (subclavian vein or axillary vein) or groin (femoral vein). See, e.g., N. Tsotsolis et al., Pneumothorax as a complication of central venous catheter insertion, ANN. TRANSL. MED., Vol. 3(3):40 (2015) (“Tsotsolis 2015”), the entire contents of which is incorporated by reference herein. Central venous catheters usually remain in place for a duration longer than other venous access devices. See, e.g., Tsotsolis 2015 at Abstract.

There are several situations that require the insertion of a central venous catheter mainly to administer medications or fluids, obtain blood tests (specifically the “central venous oxygen saturation”), and measure central venous pressure. See, e.g., Tsotsolis 2015 at Abstract. Other common inductions for centerline placement include administration of vasoactive medications, rapid resuscitation, total parenteral nutrition, and delivery of caustic medications. See, e.g., J. N. Nathwani et al., The Relationship Between Technical Errors and Decision Making Skills in the Junior Resident, J. SURG. EDUC., Vol. 73(6), pgs. e84-e90 (2016) (“Nathwani 2016”), the entire contents of which is incorporated by reference herein. Thus, the tasks performed by a central venous catheter are normally not feasible with a regular intravenous (IV) catheter inserted in the small veins of the forearm or hand. For example, continuous infusion of strong chemotherapeutic agents through a small IV line is known to cause severe tissue damage of the vessel walls, so administering chemotherapeutic drugs is one important utility of a centerline catheter because such drugs may be introduced and mixed with blood directly without contact with vessel walls.

Large surface veins such as the subclavian vein in the chest have predictable relationships to easily identifiable anatomic landmarks. For example, there are two bony landmarks that must be palpated before each attempt is made: the sternal notch and the middle to medial third of the clavicle. See, e.g., M. Kilbourne, Avoiding Common Technical Errors in Subclavian Central Venous Catheter Placement, J. AM. COLL. SURG., Vol. 208, pgs. 104-109 (2009) (“Kilbourne 2009”), the entire contents of which is incorporated by reference herein. It has also been reported that subclavian vein infection rates are lower than rates for both internal jugular and femoral catheters. See, e.g., Kilbourne 2009 at 104; see also, e.g., Nathwani 2016 at 2. Further, the subclavian vein is more accessible to the physician in trauma patients with cervical collars than the internal jugular, and the centerline catheter can be placed in the subclavian vein without disrupting airway management during the initial stage of resuscitation. See, e.g., Kilbourne 2009 at 104. Further, although the femoral vein can also be cannulated without disrupting airway management, the higher rate of infection with catheter placement in the femoral artery frequently requires the catheter to be moved to either the subclavian or internal jugular thus subjecting the patient to two separate line procedures. See, e.g., Kilbourne 2009 at 104. For the foregoing reasons, as well as the presence and use of the bony landmarks, placement of a central venous catheter into the subclavian vein is preferred over both the internal jugular and femoral veins.

The standard procedure for centerline catheter placement into the subclavian vein located at, e.g., a patient's right shoulder area is briefly summarized below.

The patient is placed in supine and Trendelenburg position with arms in adducted position and is appropriately prepped and draped. With the introducer needle attached to a syringe via a connector hub, the physician inserts the needle inferior to the clavicle and advances the needle in the cephalad (i.e., towards the head) direction by continuously aspirating with the syringe in search of the subclavian vein. The force required for inserting the needle varies with needle diameter, patient age (i.e., greater force is needed for younger patients), entry angle, and obesity. Presently, a relatively large, 18-gauge needle is used for subclavian vein puncture, which typically requires a large amount of force to push the needle through skin and tissue and into the vein. Once aspiration of blood is confirmed, the physician holds the needle firmly and removes the syringe from the connector hub.

The step of removing the syringe from the connector hub involves a certain amount of risk. For example, an amount of force—which varies depending on how the syringe tip is engaged with the connector hub and the tightness of that engagement—is needed to twist off the luer lock connection between the connector hub and the tip of the syringe. During the forceful syringe removal process, the physician must have a very firm grip on the connector hub because movement of the distal sharp needle tip can cause laceration of the vein wall. Additionally, removal of the syringe from the connector hub causes the connector hub to become open to the air and the physician must quickly place a finger over the opening immediately following syringe removal to minimize the risks of both blood loss and creation of an air embolism (i.e., introducing air into the vein).

After the syringe is removed, the physician inserts a guide wire through the opening in the connector hub vacated by the syringe and advances the guide wire through the internal lumens of both the connector hub and the needle and into the subclavian vein. The physician carefully feeds the guide wire into the vein to the desired location of the superior vena cava while leaving an appropriate length of guide wire exposed outside the connector hub.

Installing the guide wire into the vein is a delicate procedure. The physician cannot simply rush the installation of the guide wire because too much force during insertion could cause the guide wire to puncture the vein. To avoid this, the physician must slowly advance the guide wire into the vein while relying on the resistance feedback felt through the guide wire to determine when to alter the guide wire's course to prevent injury. Any additional resistance on the guide wire caused by, e.g., friction from the walls of the connector hub or needle could distort the resistance feedback felt by the physician and could thus increase the risk of injury.

Once the guide wire is installed at the desired location in the vein, the physician then holds the guide wire in place and removes the introducer needle by sliding it along the guide wire towards the distal end of the wire. To prevent blood loss during this step, the physician presses down on the skin and guide wire at the point where the need exits the skin. Before the catheter is installed, an optional “dilator” is often used to dilate the skin and venous tissue thus making room for the catheter. The dilator—fitted and threaded over the guide wire—is gently advanced into the tissue until it enters the vein. After tissue dilation, the dilator is extracted and removed from the guide wire.

Finally, the physician threads the catheter over the guide wire and advances it to the desired location inside the vein. After this step, the guide wire is no longer needed, and it is removed by pulling and exiting from the distal lumen of the catheter.

It has been reported that, in the United States, over five million catheters are placed on a yearly basis. See, e.g., Nathwani 2016 at 2. Though considered a simple procedure, centerline catheter placement is not without risk. See, e.g., Nathwani 2016 at 2. It has been reported that an estimated 15% of patients who undergo centerline placement will be confronted with at least one complication, including infection, arterial puncture, pneumothorax, hemothorax, mediastinal hematoma, and vascular thrombosis. See, e.g., Nathwani 2016 at 2. Poor technical skills, such as, e.g., poor needle angle and inadequate withdrawal of the needle prior to redirection, are reported to be the cause of these complications. See, e.g., Nathwani 2016 at 4. Due to these mistakes, physicians must often perform multiple attempts to access the vein. See, e.g., Nathwani 2016 at 4. For example, a study conducted by Michael J. Kilbourne showed that, in 86 patients, there were 357 puncture attempts with 279 failures resulting in an overall failure rate of 78.2%. See, e.g., Kilbourne 2009 at 106.

The study published by Dr. Kilbourne and colleagues highlighted five of the most common technical errors observed in the 86 patients studied. See, e.g., Kilbourne 2009 at 107. The most common technical error observed was improper needle insertion position relative to the clavicle. See, e.g., Kilbourne 2009 at 107. Kilbourne reported that in all cases the needle was inserted too closely to the bone itself, and the close proximity to the clavicle created a steep angle for cannulating the vein beneath the clavicle. See, e.g., Kilbourne 2009 at 107. Usually, this causes the needle to miss the vein in a caudal direction because the needle will not advance in between the clavicle and the first rib. See, e.g., Kilbourne 2009 at 107. Other times, according to Kilbourne, the physician would actually obtain a flash of blood but be unable to pass the guide wire distally because of significant opposing resistance caused by the guide wire striking the side wall of the vein at such a steep angle that it could not advance. See, e.g., Kilbourne 2009 at 107.

The second most common error observed by Kilbourne was insertion of the needle through the periosteum of the clavicle. See, e.g., Kilbourne 2009 at 107. Kilbourne observed that it is relatively easy to drive the needle through the periosteal layer and miss the subclavian vein anteriorly. See, e.g., Kilbourne 2009 at 107. Further, using significant force or aggressively pushing the needle can drive it through, instead of beneath, the periosteum. See, e.g., Kilbourne 2009 at 107. In other cases, according to Kilbourne, physicians attempted to bend or curve the needle around the clavicle, using the opposite hand to push down, but the needle often caught the periosteal layer during this maneuver, which caused the needle to become bent or deformed thus destroying the integrity of the internal lumen required for guide wire installation. See, e.g., Kilbourne 2009 at 107.

The third most common technical error observed by Kilbourne was taking too shallow of a trajectory of the needle. See, e.g., Kilbourne 2009 at 107. Avoiding a pneumothorax is a consideration for any physician performing subclavian vein catheterization. See, e.g., Kilbourne 2009 at 107. As a result, many physicians are concerned about the angle of the needle once it is posterior to the clavicle. See, e.g., Kilbourne 2009 at 107. This concern frequently causes the physician to mistrust the normal anatomic position of the vein and subsequently drop the needle angle too much in the coronal axis as it is passed beneath the clavicle. See, e.g., Kilbourne 2009 at 107.

Further, the two bony landmarks already mentioned (i.e., the sternal notch and the middle to medial third of the clavicle) serve as an anatomical road map leading to the correct location for needle insertion: the sternal notch serves as the reference point for needle directionality and the middle third of the clavicle provides the starting point for skin puncture. See, e.g., Kilbourne 2009 at 107. Yet, the fourth most common technical error observed by Kilbourne was improper or inadequate anatomic landmark identification. See, e.g., Kilbourne 2009 at 107.

The fifth most common technical error observed by Kilbourne was aiming the needle too cephalad. See, e.g., Kilbourne 2009 at 107. According to Kilbourne, part of the motivation to do this lies in the fact that mechanical complications like pneumothorax are a significant concern. See, e.g., Kilbourne 2009 at 107. Consequently, the urge to aim cephalad and away from the pleural apex can cause the physician to miss the vein superiorly. See, e.g., Kilbourne 2009 at 107.

The current and conventional device used to perform a centerline catheter placement into the subclavian vein is a relatively long, straight introducer needle attached via a connector hub to a 10-cc syringe. In the current standard-of-care, the longitudinal axes of the straight needle, straight connector hub, and straight syringe are all aligned together to create one long straight needle/hub/syringe assembly. In this set-up, the syringe and/or the connector hub serve as a handle for the physician to grip and operate the introducer needle. Examples of such longitudinally straight needle/syringe assemblies may be found in, e.g., U.S. Pat. Nos. 5,290,244 (Moonka), 5,735,813 (Lewis), and 6,371,944 (Liu et al.), the entire contents of each of which is incorporated by reference herein.

Ideally, during the procedure, the introducer needle should seek and advance toward the subclavian vein and stay substantially inside the vein lumen along the vein's axis. But the straight longitudinal design of the current and conventional needle/syringe assemblies imposes a severe geometrical limitation on direction of needle advancement because of the way in which the needle must be held during the procedure and because of certain anatomical obstacles. Even when the vein is penetrated, the needle is likely to assume a large blunt angle relative to the vein axis and may easily penetrate through the vein wall in the transverse direction. Thus, the use of a needle/syringe assembly with such a straight longitudinal design severely hinders successful subclavian vein cannulation and contributes to the several of the most common technical errors observed by Kilbourne. Because the subclavian vein is close to the lung and arteries in a human patient, any inadvertent or incorrect needle movement may result in serious and dangerous complications.

The risk of serious injury is further increased during guide wire installation when the physician removes the syringe and quickly places a thumb over the resulting opening to minimize blood loss and avoid air introduction. Even still, a certain amount of blood loss occurs after the physician removes the thumb to allow installation of the guide wire. The blood loss, however, may cause an urgency in the physician to perform the installation as quickly as possible, which exacerbates the risk by leading to a greater incidence of technical errors. Certain conventional needle/syringe assembly devices attempt to minimize this problem by providing a separate guide wire insertion port on the connector hub. In some of these devices, however, the insertion port is not hermetically sealed, which prevents the step of aspirating blood into the syringe to test whether the needle has found the vein and greatly increases the risk of introducing air into the vein. In other devices, the separate insertion port is located at a sharp angle relative to the needle and/or is too long, which complicates the installation of the guide wire by, e.g., causing the guide wire to drag along the internal surfaces of the needle and connector hub. In other cases, the sharp angle and elongated insertion port causes the connector hub to be inoperable if, e.g., the angle is too sharp to maneuver the guide wire around and into the internal lumen of the introducer needle.

Some references discuss an angled or bent needle/hub/syringe assembly but fail to contemplate the necessity of hermetically sealing the whole assembly to allow for aspirating blood into the syringe. Importantly, aspirating blood into the syringe is a confirmatory signal that the needle is in the vein. Further, leaving the separate insertion port open to the air allows air to enter the connector hub and introducer needle and thus greatly increases the risk of dangerous venous air embolisms. Accordingly, without being hermetically sealed, the angled or bent needle/hub/syringe assemblies are not only inoperable; they are dangerous. Further, the insertion port component of such assemblies is too long, and the angle of the longitudinal axis of the insertion port relative to the longitudinal axis of the needle is too sharp, which means a physician would have difficulty maneuvering the guide wire around that angle and into the needle lumen. Even if the physician were successful in doing so, however, the increased friction resistance on the guide wire would also increase the risk of injury to the patient.

Accordingly, the use of a needle/hub/syringe assembly with a straight longitudinal design in the current standard-of-care centerline catheter placement procedures has been fraught with complications leading to pneumothorax, hemothorax, arterial and thoracic punctures, venous laceration, brachial plexus and other injuries. Thus, the use of longitudinally straight needle/hub/syringe assemblies to place a centerline catheter via the subclavian vein significantly compromises patient safety, and physicians performing the placement are, at times, inflecting serious and dangerous injuries on the patient, which creates fear among physicians performing the procedure.

For example, it has been reported that the overall complication rate for center line catheter placement procedures is 15%, ranging from 5% to 19%. See Tsotsolis 2015 at pg. 1 or 10. Tsotsolis 2015 reports the following rates of complications for subclavian vein center line placement procedures: mechanical incidence (6.2% to 10.7%); arterial puncture (3.1% to 4.9%); hematoma (1.2% to 2.1%); and pneumothorax (0.45% to 3.1%). Tsotsolis 2015 at Table 1. But it is believed that the number of complications resulting from centerline catheter placement procedures is grossly under-reported. Indeed, it is believed that the literature only captures the complications that occur at major hospitals while the complications that occur at smaller, more regional hospitals remain unreported. Further, the literature typically fails to report aborted placement attempts, which often cause the physician to use a less desirable placement site. Yet, despite the reported number of serious injuries, longitudinally straight needle/hub/syringe assemblies remain the standard of care throughout the U.S. and the world.

While there are three major sites for insertion of centerline catheters—the subclavian, jugular and femoral sites—the subclavian site has the most advantages despite the higher risk of pneumothorax with use of the standard longitudinally straight introducer needle/hub/syringe assemblies. Because the assembly is straight, fear of causing pneumothorax and other complications are the main reason for selecting the alternate, less advantageous femoral and jugular sites.

Some of the complications associated with syringe removal, vessel laceration, blood loss and air embolisms may be averted by replacing the conventional syringe by a unique Raulerson syringe as described in, e.g., U.S. Pat. No. 4,813,938, the entire contents of which is incorporated by reference herein. The Raulerson syringe is constructed with a hollow piston plunger that slides over a metal cannula, which extends between both ends of the syringe receptacle barrel and is designed to eliminate the extra step of syringe removal necessary for guide wire placement. The metal cannula has a small hole on the side wall near the proximal end enabling blood to emerge when aspiring with the sliding piston plunger. Additionally, the cannula allows insertion of the guide wire through the opening located at the distal part of the plunger. The guide wire enters the hollow cannula and advances forward directly through the connector hub into the hollow needle lumen. Because the Raulerson syringe is not separated, the introducer needle, connector hub, and syringe stay together as an integral unit, which is much easier to secure and stabilize than in the case with syringe removed. Thus, in using a Raulerson syringe, the guide wire can be placed safely and with great ease and without risk for vein laceration. Further, during the guide wire insertion step, risks of blood loss and air aspiration from an open hub are reduced or eliminated. Once it is confirmed that the guide wire has entered the subclavian vasculature, the needle along with the attached Raulerson syringe can be removed together from the open end of the guide wire. The remainder of the catheter placement procedure is performed in the usual fashion.

Importantly, however, the Raulerson syringe, connector hub, and needle assembly are still aligned along the same straight longitudinal axis, which is the leading cause of several of the most common technical errors and injuries that occur during the procedure. Further, the complicated design of the Raulerson syringe makes it expensive and impractical.

Other devices have been reported that use shaped, curved or bent needles. But the bent needles are exclusively used for fluid introduction, aspiration, and sample retraction, not for inserting a guide wire into the subclavian vein, which requires a more specialized needle. For example, in fabricating these bent or curved needles there is no requirement for maintaining a suitably open (i.e., hollow) lumen configuration to accommodate a smooth-sliding guide wire to reach the vena cava of a human heart. The applications of these curved or bent needles include administering a fluid or local anesthetic into a ligamentary tissue of a tooth, oral cavity, spinal intrathecal space, retinal blood vessel, eye, wound, blood vessel, human face, ear canal, and many other body locations. None of these curved or bent needles are usable or are intended for placement of subclavian vein catheters. That is, one problem with the curved or bent needles is that the curve or bend weakens the structural integrity of the needle at the curve or bend which may cause the needle to collapse when the physician attempts to puncture the skin and vein. Further bending or curving the needle may distort the needle's internal lumen thereby causing undesirable resistance on the guide wire as it is threaded through the needle lumen and into the vein, which can also lead to injury and other complications.

Thus, there remains a need in the art for a needle/hub/syringe assembly that alleviates the dangers and complications of the present-day straight needle/hub/syringe assembly designs. Additionally, there remains a need in the art for a specially formed connector hub that facilitates alleviating the technical errors associated with centerline catheter placement into the subclavian vein, and that also provides a hermetically sealed insertion port that allows blood to be aspirated into the syringe once the needle is in the vein and prevents air from entering the vein, and that allows reduced friction resistance on the guide wire during installation.

### SUMMARY OF THE INVENTION

The object of the present invention is to provide a device that reduces the risks involved with placing a centerline catheter into the subclavian vein. Generally, the risks are reduced because the device of the present invention mimics desirable anatomical angles at the site of insertion in the human patient's shoulder and is easier to use than the widely-used standard-of-care longitudinally straight introducer needle/hub/syringe assemblies. Further, the risks of placing a centerline catheter into the subclavian vein are reduced because the device of the present invention has a structure that facilitates easy insertion of the guidewire with minimal frictional resistance and minimal risk of forming air embolisms.

It is a further object of the present invention to provide a device that reduces the rates of technical errors and placement failures that occur during the placement of a centerline catheter into the subclavian vein. By reducing the rates of technical errors and placement failures, centerline catheters may be secured in place quicker when using the device of the present invention than when using the conventional longitudinally straight introducer needle/hub/syringe assemblies. As a result, the risk of injury is reduced, and potentially life-saving medical attention can be administered quicker as well.

It is another object of the present invention to provide a kit and/or system for placing a centerline catheter into the subclavian vein of a patient in need thereof, the kit comprising an introducer needle, a syringe, and a connector hub as described in the below detailed description.

It is another object of the present invention to provide a method for using an assembly comprising an introducer needle, connector hub, and syringe that is described in the below detailed description. It is yet another object of the present invention to provide a method of using a specially formed connector hub as described in the below detailed description. It is a further object of the present invention to provide a method of placing a centerline catheter into the subclavian vein of a human patient in need thereof using an assembly comprising an introducer needle, connector hub, and syringe as described in the below detailed description. It is a still further object of the present invention to provide a method of placing a centerline catheter into the subclavian vein of a human patient using the specially formed connector hub as described in the below detailed description. The methods of the present invention enable accurate, reliable, and safe access of the subclavian vein via the infra-clavicular site.

It is another object of the present invention to provide a method of manufacturing an assembly comprising an introducer needle, connector hub, and syringe that is described in the below detailed description. It is also an object of the present invention to provide a method of manufacturing a connector hub that is described in the below detailed description. In some embodiments, e.g., the connector hub according to the present invention may be manufactured by injection molding. In other embodiments, the connector hub according to the present invention may be manufactured by 3D printing. Other manufacturing methods are also contemplated, such as, e.g., extrusion blow molding, injection blow molding, and vacuum casting.

One object of the present invention is to provide a connector hub for a needle assembly for placing a centerline catheter into the subclavian vein of a patient in need thereof, said connector hub comprising a first portion located at a first end section of the connector hub, said first portion comprising a first opening, a first passage fluidly connected to said first opening, and a first longitudinal axis, a second portion located at a second end section of the connector hub opposite said first end, the second portion comprising a second opening, a second passage fluidly connected to said second opening, and a second longitudinal axis, a medial portion located at a middle section of the connector hub between said first end section and said second end section, said medial portion comprising a medial passage fluidly connected with said first passage and said second passage, and a medial longitudinal axis, wherein said medial longitudinal axis is arranged at an angle relative to said first longitudinal axis of said first portion, and wherein said medial longitudinal axis is arranged at an angle relative to said second longitudinal axis of said second portion, and at least one insertion port located on said medial portion, wherein said insertion port comprises a first insertion port opening, an insertion port passage fluidly connected to said medial passage, and an insertion port longitudinal axis, wherein said first insertion port opening is hermetically sealed and is configured to accept a catheter guide wire, and wherein said insertion port longitudinal axis is arranged at an angle relative to said medial longitudinal axis.

### BRIEF DESCRIPTION OF THE DRAWINGS

The subject matter regarded as the invention is particularly pointed out and distinctly claimed in the concluding portion of this specification. The invention, however, both as to organization and method of operation, together with objects, features, and advantages thereof, may best be understood by reference to the following detailed descriptions when read with the accompanying drawings in which:

FIG. 1 is a three-dimensional depiction of a representative connector hub according to certain aspects of the present invention;

FIGS. 2A and 2B are cross-sectional depictions of a side view and a top view, respectively, of a representative connector hub according to certain aspects of the present invention;

FIG. 2C is a cross-sectional depiction of a side view of a representative connector hub according to certain aspects of the present invention;

FIG. 2D is a three-dimensional depiction of a side view of a representative connector hub according to certain aspects of the present invention;

FIG. 2E is a transparent three-dimensional depiction of a side view of a representative connector hub according to certain aspects of the present invention;

FIG. 3A is a cross-sectional depiction of a side view of a representative connector hub according to certain aspects of the present invention;

FIG. 3B is a three-dimensional depiction of a side view of a representative connector hub according to certain aspects of the present invention;

FIG. 3C is a transparent three-dimensional depiction of a top view of a representative connector hub according to certain aspects of the present invention;

FIGS. 4A and 4B are cross-sectional depictions of a side view and a top view, respectively, of a representative connector hub according to certain aspects of the present invention;

FIGS. 5A and 5B are cross-sectional depictions of a side view and a top view, respectively, of a representative connector hub according to certain aspects of the present invention;

FIG. 5C is a transparent three-dimensional depiction of a top view of a representative connector hub according to certain aspects of the present invention;

FIG. 6 is a cross-sectional depiction of a side view of a representative connector hub according to certain aspects of the present invention;

FIGS. 7A and 7B are cross-sectional depictions of a top view and a side view, respectively, of a representative connector hub according to certain aspects of the present invention;

FIG. 7C is a three-dimensional depiction of a representative connector hub according to certain aspects of the present invention;

FIG. 7D is a transparent three-dimensional depiction of a side view of a representative connector hub according to certain aspects of the present invention;

FIG. 8 is a top view and side views of a representative membrane according to certain aspects of the present invention;

FIG. 9 is a top view and side views of a representative membrane according to certain aspects of the present invention; and

FIGS. 10A, 10B, and 10C are top views of representative membranes according to certain aspects of the present invention.

It will be appreciated that, for simplicity and clarity of illustration, elements shown in the figures have not necessarily been drawn to scale. For example, the dimensions of some of the elements may be exaggerated relative to other elements for clarity. Additionally, the many features of any one embodiment shown in a figure should not be considered independent and separate from the features of an embodiment shown in another figure, and it is conceivable that features of any one embodiment may be combinable with another. Further, where considered appropriate, reference numerals may be repeated among the figures to indicate corresponding or analogous elements. Moreover, the arrows and braces used to point to different parts and portions of the embodiments shown in the figures are approximate only and should not be considered limiting in any way.

### DETAILED DESCRIPTION OF THE INVENTION

In the following detailed description, numerous specific details are set forth in order to provide a thorough understanding of the invention. It will be understood by those of ordinary skill in the art, however, that the present invention may be practiced without these specific details. In other instances, well-known methods, procedures, and/or components have not been described in detail so as not to obscure the present invention. Further, it will be understood by those of ordinary skill in the art that the invention(s) disclosed herein should not be limited to any one specific embodiment and that different embodiments may be contemplated, including embodiments that contain all or part of the specifically described embodiments or that contain a mixture of components of the several specific embodiments described herein.

Further, the present invention is described in the context of exemplary embodiments. The scope of the invention, however, is not limited to the particular examples and embodiments described in the specification. Rather the specification merely reflects certain embodiments and serves to illustrate the principles and characteristics of the present invention. Those skilled in the art will recognize that various modifications and refinements may be made without departing from the spirit and scope of the invention.

During any subclavian vein catheter placement procedure, the patient would normally be positioned lying supine with arms alongside the body, in a 15 to 30 degrees head down Trendelenburg position and with patient's head turned to the contra-lateral side of the needle puncture point. The Trendelenburg position is used to reduce venous blood loss during guide wire introduction, to enhance blood fill and vein distension, and to minimize the risk of an air embolism. Additionally, a substantial scapula wedge with rolled towels is sometimes required to make the clavicle more prominent. Further, for infra-clavicular cannulation, advancing the introducer needle very close to, and in parallel with, the skin surface of the patient's chest is the most important requirement because—when such a requirement is satisfied—the introducer needle would most certainly puncture the subclavian vein at the sub-clavicular site with a much-reduced risk of pneumothorax and other injuries. As such, the optimum needle path is one that hugs the skin surface. Notably, with the present invention, positioning the patient in the Trendelenburg position is an optional step, as some patients do not tolerate such a position.

To date, a long straight needle (about 2.5 inches in length), rather than a short one, is commonly used because the needle length helps to maintain as shallow of an angle as possible relative to the horizontal plane of the skin surface. The conventional, standard-of-care device for performing a subclavian vein catheter placement is a straight 10-cc syringe connected to a straight, long introducer needle that is connected to the syringe via a straight connector hub. When using the latter standard-of-care straight needle/hub/syringe assemblies, the physician typically holds the syringe barrel to maneuver the attached introducer needle and perform the procedure. But holding the conventional straight assemblies in this way makes it difficult to maintain the introducer needle in the required position parallel to the surface of the patient's skin. This difficulty is exacerbated by the force that is required from the physician to puncture the skin and advance the needle through the tissue around the patient's clavicle because the way in which the physician must typically grasp the assembly to provide enough force also interferes with maintaining the introducer needle parallel to the surface of the patient's skin. Further, after the skin is punctured, the conditions required for a successful centerline catheter placement in the subclavian vein include moving the introducer needle horizontally in parallel with the surface of the patient's chest and in the direction toward the middle third of the clavicle. These conditions are not satisfied, however, when the physician uses the standard-of-care straight needle/hub/syringe assemblies. The syringe size and physician's hand holding the syringe over the patient's chest each imposes a limit on the optimal needle approach angle and path.

Further, some patients are unable to tolerate a Trendelenburg position and some elderly and obese patients may not tolerate a scapula wedge placement. Other physical obstacles to successful catheter placement may include, e.g., the body build, patient obesity, distorted anatomy, deformity of chest wall and neck, wide area of the clavicle, as well as variation of the patient's position and natural body landmarks. These physical obstacles may greatly hinder placement of a centerline catheter into the subclavian vein because they hinder proper insertion of the longitudinally straight introducer needle assemblies close to, and in parallel with, the skin surface of the patient's chest. That is, the physical obstacles may affect the introducer needle's entry angle, skin puncture location, needle approach, and direction of advancement. Indeed, the proper needle path may not be obtainable with a straight needle, especially for obese patients with large and bulging humeral mass and for elderly patients with fused or stiff shoulder muscle in whom a scapula wedge with rolled towels is either not tolerated or is ineffective.

Due to these difficulties, conventional introducer needles often puncture the skin and advance subcutaneously at an angle relative to the skin surface of the patient's chest. Since the subclavian vein is located just beneath the middle third of the clavicle, the use of conventional standard-of-care straight needle assemblies, which must be advanced at an angle rather than parallel to the surface of the patient's skin (with patient in supine position), typically results in technical errors and unsuccessful procedures. Also, since the apex pulmonic (i.e., the dome of the pleura of the lung) is located just posterior to the subclavian vein at the sub-clavicular location, there is a greater chance for causing pneumothorax and hemothorax injury, which require thoracostomy and other emergency procedures. With these considerations in mind, judicious selection of introducer needle puncture point, angle, needle advancement direction, and subcutaneous needle path are required to successfully gain entry into the subclavian vein.

One aspect of the present invention is to provide a needle assembly for use during a centerline catheter placement, especially the placement of a centerline catheter into the subclavian vein of a patient. The needle assembly comprises three parts: an introducer needle, a syringe, and a connector hub that fluidly connects the introducer needle with the syringe. The connector hub has four distinct portions all fluidly connected. The introducer needle is located at a first portion of the connector hub. The first portion is located at one terminal end of the connector hub and contains an internal passage that fluidly connects the first portion to the rest of the connector hub. The syringe is located at a second portion of the connector hub. The second portion is located at the other terminal end of the connector hub opposite the first end and contains an internal passage that fluidly connects the second portion with the rest of the connector hub. A medial (i.e., middle) portion of the connector hub serves to connect the first and second portions of the connector hub together. The medial portion also contains an internal passage that is fluidly connected with the internal passages of the first and second portions. Because each of the first, second, and medial portions are fluidly connected, the connector hub also serves to fluidly connect the introducer needle with the syringe.

Further, in one aspect of the invention, the first portion of the connector hub is arranged at an angle (e.g., about 10-50 degrees, about 15-45 degrees, or about 20-40 degrees) relative to the medial portion. In another aspect of the invention, the second portion of the connector hub is arranged at an angle (e.g., about 10-50 degrees, about 15-45 degrees, or about 20-40 degrees) relative to the medial portion.

During a centerline catheter placement procedure, a physician may grasp the connector hub assembly or the syringe barrel to insert the introducer needle into a patient's vein and check for vein entry by aspirating with the syringe. During the procedure, the angled arrangement of the portions of the connector hub according to the present invention alleviates the difficulties faced when using the conventional straight needle/hub/syringe designs because it not only mimics anatomical features of a patient's clavicle but also allows the physician to grasp the assembly in a way that does not cause the physician's hand to be an obstacle.

Additionally, the medial portion of the connector hub also includes an insertion port. The insertion port includes an opening that is hermetically sealed. The insertion port also includes a passage that is fluidly connected with the medial portion's internal passage and, thus, the rest of the connector hub, including the introducer needle and the syringe. The insertion port is arranged at an angle (e.g., about 10-50 degrees, about 15-45 degrees, or about 20-40 degrees) relative to the medial portion, not necessarily the same angle or in the same radial direction as the connector hub's first and second portions. During a centerline catheter placement, a guide wire may breach the hermetic seal on the insertion port and extend along the internal passages of the connector hub until it extends through the internal lumen of the introducer needle and into the vein.

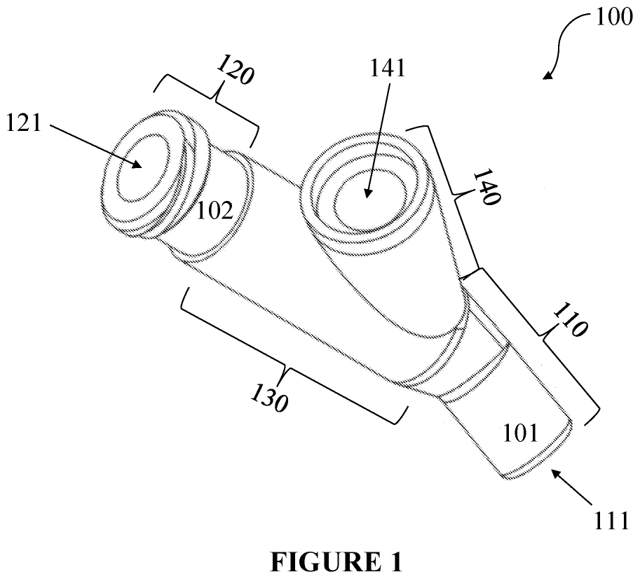

Accordingly, the present invention discloses device(s), system(s), kit(s), and method(s) that reduce the risks and alleviates many, if not all, of the difficulties, technical errors, and failures associated with the placement of a centerline catheter in the subclavian vein. The device(s), system(s), kit(s), and method(s) for safe and efficacious placement of a centerline catheter into the subclavian vein are described with reference to the embodiments shown in FIGS. 1, 2A-2E, 3A-3C, 4A-4B, 5A-5C, 6, 7A-7D, 8, 9, and 10A-10C.

The device(s), system(s), kit(s), and method(s) of the present invention are based on the use of a straight introducer needle aligned at an angle relative to the longitudinal axis of the syringe and/or aligned at an angle relative to the guide wire insertion port. Specifically, the device(s), system(s), kit(s), and method(s) of the present invention are based on a connector hub that connects the straight introducer needle and straight syringe together, wherein the connector hub has one or more portions aligned at an angle relative to other portions of the connector hub. Further, the device(s), system(s), kit(s), and method(s) of the present invention include a connector hub having at least two branches or ports—a first branch/port configured to engage with a syringe; and a second branch/port configured to allow easy entry of a catheter guide wire without the need for removing the syringe, the second branch/port comprising a seal (e.g., a hermetic seal). Due to installation of the second branch/port comprising the seal, the catheter guide wire may be installed into the subclavian vein without the necessity of removing the syringe, which is tightly engaged with the first branch/port by strong friction fit.

Certain aspects of the present invention exhibit an introducer needle that maintains a fixed angle between, e.g., about 10 to 50 degrees, about 15 to 45 degrees, or about 20 to 40 degrees relative to a longitudinal axis of portions of the connector hub and/or a longitudinal axis of the syringe. The syringe may function as a handle for the physician to grasp and hold in order to maneuver the introducer needle to an optimal location for skin puncture, entry angle, and advancement direction after entering the skin. Because the introducer needle is aligned at an angle relative to, e.g., portions of the connector hub and/or syringe, the physician's hand gripping the syringe no longer limits or blocks the needle entry angle or affects the needle movement or advancement as it does with the conventional standard-of-care straight needle/hub/syringe assemblies.

Further, physical obstacles such as, e.g., a bulging humeral mass or a cavity formed by a stiff shoulder muscle no longer hinder proper needle introduction. The majority of patients requiring central line catheterization are critically ill and they often cannot tolerate being placed on a hard surface to use a scapula wedge. But the device(s), system(s), kit(s), and method(s) of the present invention eliminate the need for a scapula wedge with rolled towels and allows the critically ill patient to be placed on either a soft or hard surface depending on patient preference or other medical considerations.

Additionally, with the device(s), system(s), kit(s), and method(s) of the present invention, the physician can keep the needle in the horizontal plane relative to the patient's chest and advance the needle medially for easy and safe subclavian vein puncture. Thus, the angular nature of the device(s), system(s), and kit(s) of the present invention provides the physician with an unencumbered maneuverability for angle of skin puncture, direction of needle advancement following skin entry, and increased control of the needle's position relative to the skin surface. Contrary to the conventional straight needle/hub/syringe assemblies, the device(s), system(s), and kit(s) of the present invention, which comprise a straight introducer needle arranged at an angle relative to a longitudinal axis of the syringe and/or relative to a longitudinal axis of a portion of the connector hub, may be easily positioned and maneuvered to avoid anatomical obstacles thus reducing the risk of dangerous complications and injuries.

For example, with the use of the device(s), system(s), kit(s), and method(s) of the present invention the straight needle shaft can be maintained at a horizontal position after puncturing the skin, and it can hug the internal skin surface. In this way, pneumothorax and other similar injuries can be avoided. For example, the introducer needle used with the device(s), system(s), kit(s), and method(s) of the present invention can easily be inserted and manipulated to advance only along paths in parallel with the pleura surface with a reduced risk of causing a pneumothorax episode. The post-puncture position and angle achievable by the present invention is not possible with the current standard-of-care straight needle/hub/syringe assemblies because the physician's hand holding the syringe of the conventional assemblies must stay above the chest, thus placing a limit on the incline angle of the straight needle relative to the chest surface.

Additionally, another advantage of the device(s), system(s), kit(s), and method(s) of the present invention is the insensitivity of the invention to the needle insertion location. Because the straight needle is arranged at an angle relative to the syringe and/or connector hub, it can continuously hug the internal skin surface in all directions without concern for pneumothorax and can be safely directed to the infra-clavicular subclavian vein site from any initial skin puncture location. Contrary to the present invention, the needles of the conventional straight assemblies are often inserted too close to the clavicle, which creates a steep angle that causes the needle to miss the vein in a caudal direction. But this problem is avoided with the present invention, which involves a straight needle arranged at an angle relative to the longitudinal axis of the syringe and/or a longitudinal axis of a portion of the connector hub, and the present invention allows an optimal angle to be used for any non-ideal skin puncture locations.

With the use of the device(s), system(s), kit(s), and method(s) of the present invention, many common errors encountered by the conventional, standard-of-care straight needle/hub/syringe assemblies can be avoided. For example, inserting the needle through the periosteal layer of the clavicle is one of the most common technical errors that occurs from using the conventional assemblies. Many physicians poke the clavicle with the needle tip to locate the vein beneath the clavicle, and it is a common occurrence to drive the needle through the periosteum and miss the subclavian vein anteriorly. But, with the use of the present invention, this risky, tissue damaging, and debilitating “walking down the clavicle” approach can be avoided.

Further, during the popular entry approach to the right subclavian vein, a physician holding the device(s), system(s), and/or kit(s) of the present invention can easily puncture the skin at a few centimeters caudad to the clavicle at the junction of the middle and medial thirds of the clavicle. Because the introducer needle is aligned at an angle relative to the longitudinal axis of the syringe and/or to a longitudinal axis of a portion of the connector hub, the physician's hand and syringe remain above the chest and skin surface of the patient such that the needle shaft can be moved along the horizontal transverse plane, parallel to the skin surface of the chest and, most importantly, parallel to the pleura surface to avoid a pneumothorax event. Since physician's hand is no longer an obstacle for free movement of the needle, the introducer needle can enter the subclavian vein substantially inside the lumen along the axial direction.

These advantages are made possible only by use of device(s), system(s), kit(s), and method(s) of the present invention. In contrast, the conventional and standard-of-care straight needle/hub/syringe assemblies impose a severe geometrical limitation on the direction of needle advancement. For example, even when the vein is penetrated, the needle is likely to assume a large blunt angle relative to the vessel axis and may easily penetrate through the vein walls in the transverse direction. Thus, use of conventional straight assemblies severely hinders successful subclavian vein cannulation and is the cause of several recurring technical errors that increase failure rate. Further, because the subclavian vein is close to the lung and arteries, any inadvertent or incorrect needle movement may result in serious injuries and complications.

In one aspect, the invention provides a connector hub for an introducer needle assembly. In another aspect, the invention provides a needle assembly comprising an introducer needle, a connector hub, and a syringe. In the latter embodiment the connector hub is configured to connect the introducer needle with the syringe. In certain other embodiments, the present invention provides a kit or a system comprising an introducer needle, a connector hub, and a syringe as described herein. The kit may further include a guide wire.

The introducer needles that may be used with certain embodiments of the present invention are hollow, large gauge needles that are well-known in the art of centerline catheter placement procedures. For example, the introducer needles that may be used with the present invention may be, in some embodiments, approximately 2.5 inches long and comprise an internal lumen with a diameter large enough to allow a guide wire to be threaded through the lumen. In preferred embodiments, the introducer needle is equipped with a connector portion configured to connect the needle to a syringe or connector hub. Generally, introducer needles that may be used with the present invention are, e.g., 18-gauge XTW (i.e., extra thin wall) tubing having a regular beveled tip, a length of about 2.5 inches±about 0.10 inches an internal lumen diameter of about 0.042 inches±about 0.001 inches, an outside diameter of about 0.050 inches±about 0.0005 inches, and manufactured from a hypodermic needle stock material such as, e.g., 304 stainless steel. The introducer needle may also have an acid-passivated and polished surface treatment.

The syringes that may be used with certain embodiments of the present invention are well-known in the art of centerline catheter placement procedures. In general, the syringes that may be used with the present invention may include, in some embodiments, an elongated hollow receptacle barrel, a plunger acceptable within the hollow receptacle barrel, and a connector portion configured to connect the syringe to an introducer needle assembly or a connector hub. In preferred embodiments, the syringe comprises a luer taper assembly (i.e., an assembly per ISO 80369-7, 2016) comprising a male luer fitting that is configured to engage with a female luer fitting located on either an introducer needle assembly or a connector hub.

In certain embodiments, the device of the present invention comprises a connector hub for an introducer needle assembly wherein the connector hub comprises a distal end and a proximal end. In certain embodiments, the distal end and the proximal end of the connector hub are arranged in different planes such that the distal end and proximal end are not aligned along a straight longitudinal axis.

In certain embodiments of the present invention, the connector hub is generally cylindrical in shape or branched cylindrical shape, although other shapes of the connector hub are contemplated such as, e.g., ellipsoidal, ovoid, or conical. It is contemplated, however, that the connector hub according to certain embodiments of the present invention may be comprised of portions wherein each portion is a different shape. For example, one portion of the connector hub may be a square, cube, or rectangular shape while other portions may be a cylindrical, ellipsoidal, ovoid, or conical shaped.

In certain embodiments, the connector hub of the present invention may be manufactured from a translucent, transparent, or otherwise clear material. The translucent, transparent, or otherwise clear material allows the display of blood, which allows the physician to view the blood and discriminate between venous and arterial blood by visualizing the color differential, visualizing pulsating arterial versus non-pulsating venous blood, or visualizing fast-moving arterial blood versus slow-emerging venous blood. Manufacturing the connector hub of the present invention with such materials quickens the procedure for placing a centerline catheter by enabling early and rapid observation of the blood merging from the needle into the connector hub and onward to the syringe.

In certain embodiments of the present invention, the connector hub may be manufactured from a polycarbonate material such as, e.g., Covestro AG's MAKROLON® 2558 polycarbonate, which is one example of a translucent, transparent, or otherwise clear material that is suitable for use as a material for manufacturing the connector hub. In some embodiments, e.g., the connector hub according to the present invention may be manufactured by injection molding. In other embodiments, the connector hub according to the present invention may be manufactured by 3D printing. Other manufacturing methods are also contemplated, such as, e.g., extrusion blow molding, injection blow molding, and vacuum casting.

In certain embodiments, the connector hub of the present invention is manufactured such that the internal volume of the connector hub is minimized to the extent possible. For example, the connector hub of the present invention may have a low-volume configuration. The low, or minimized, internal volume of the connector hub enables more efficient and rapid detection of aspirated blood—either arterial blood or venous blood—while reducing the force and movement needed to aspirate blood by operating the syringe plunger. That is, the reducing the internal volume of the connector hub reduces the force required to create a negative pressure within the assembly in order to draw blood through the needle, into the connector hub, and onward to the syringe.

In some embodiments, for example, the internal volume of a connector hub according to certain aspects of the present invention may be between about 0.03 ml to about 0.08 ml, between about 0.035 ml to about 0.075 ml, between about 0.04 ml to about 0.07 ml, and between about 0.045 ml to about 0.065 ml, while in the internal volume of the connector hub may be between about 0.0625 ml to about 0.065 ml or about 0.0635 ml in some preferred embodiments. Other volumes for the low-volume configuration of the connector hub may be contemplated. Generally, the foregoing volumes of the low-volume connector hub excludes the internal volume of the insertion port lumen because the hermetic seal of the insertion port prevents blood from entering the insertion port lumen when aspirating blood by operating the syringe.

In certain embodiments, the connector hub of the present invention comprises a first portion having a first opening, a first passage and a first longitudinal axis. Generally, the first opening is an opening that leads into the first passage, the first passage being a hollow lumen. In certain embodiments, the length of the first portion of the connector hub may be between about 0.15 to about 0.50 inches, about 0.20 to about 0.45 inches, about 0.25 to about 0.40 inches, or about 0.30 to about 0.35 inches. In other embodiments, the length of the first portion of the connector hub may be about 0.15, 0.20, 0.25, 0.30, 0.35, 0.40, 0.45, or 0.50 inches. In certain embodiments the length of the first portion of the connector hub is about 0.25 or about 0.40 inches. Other lengths are contemplated, however. Generally, the overall length of the connector hub of the present invention, including the first portion, should be minimized. In certain embodiments, the first portion of the connector hub of the present invention further comprises a first distal end and a first proximal end. In some embodiments, the first opening of the first portion is located at the first distal end.

In some embodiments, the first portion is configured to engage with an introducer needle useful for performing a centerline placement procedure. In certain embodiments, when the first portion is engaged with an introducer needle, the first longitudinal axis of the first portion is arranged relative to a longitudinal axis of the introducer needle at an angle of about zero degrees, although other angles are contemplated. In some embodiments, the first portion of the connector hub of the present invention may comprise a male luer fitting that is configured to engage with a female luer fitting on an introducer needle. For example, the first opening at the first distal end of the first portion may be formed as a male luer fitting. In other embodiments, however, an introducer needle may be integrally formed with, and/or embedded within, the first portion of the connector hub of the present invention. In all contemplated embodiments, when the first portion of the connector hub is engaged with the introducer needle, the introducer needle's internal lumen and the first passage are fluidly connected.

In certain embodiments, the connector hub of the present invention comprises a second portion having a second opening, a second passage and a second longitudinal axis. Generally, the second opening is an opening that leads into the second passage, the second passage being a hollow lumen. In certain embodiments, the length of the second portion of the connector hub may be between about 0.10 to about 0.40 inches, about 0.15 to about 0.35 inches, or about 0.20 to about 0.30 inches. In other embodiments, the length of the second portion of the connector hub may be about 0.10, 0.15, 0.20, 0.25, 0.30, 0.35, or 0.40 inches. In other embodiments, the length of the second portion of the connector hub is, e.g., about 0.30 inches. Other lengths are contemplated, however. Generally, the overall length of the connector hub of the present invention, including the second portion, should be minimized. In certain embodiments, the second portion of the connector hub of the present invention further comprises a second distal end and a second proximal end. In some embodiments, the second opening of the second portion is located at the second proximal end.

In some embodiments, the second portion is configured to engage with a syringe useful for performing a centerline placement procedure. In certain embodiments, when the second portion is engaged with a syringe, the second longitudinal axis of the second portion is arranged relative to a longitudinal axis of the syringe at an angle of about zero degrees, although other angles are contemplated. In certain embodiments, the second portion of the connector hub of the present invention may comprise a female luer fitting that is configured to engage with a male luer fitting on a syringe. In other embodiments, however, a syringe may be integrally formed with, and/or embedded within, the second portion of the connector hub of the present invention. In all contemplated embodiments, when the second portion of the connector hub is engaged with the syringe, the syringe's hollow receptacle barrel and the second passage are fluidly connected.

In certain embodiments, the first distal end of the first portion is the same as the distal end of the connector hub of the present invention. Further, in certain embodiments, the second proximal end of the second portion is the same as the proximal end of the connector hub of the present invention.

In certain embodiments, the connector hub of the present invention comprises a medial portion arranged between the first portion and the second portion, wherein the medial portion comprises at least two medial openings, a medial passage, and a medial longitudinal axis. Generally, the medial passage is a hollow lumen that is fluidly connected with both the first passage of the first portion as well as the second passage of the second portion. The medial portion comprises a medial distal end and a medial proximal end. In certain embodiments of the connector hub of the present invention, the first portion is located at the medial distal end of the medial portion. In certain embodiments of the connector hub of the present invention, the second portion is located at the medial proximal end of the medial portion.

In certain embodiments of the connector hub of the present invention, the medial longitudinal axis is arranged relative to the first longitudinal axis of the first portion at an angle between about zero degrees and about 90 degrees. In other embodiments, the medial longitudinal axis is arranged relative to the first longitudinal axis of the first portion at an angle between about 10 and about 50 degrees; about 15 and about 45 degrees; and about 20 and about 40 degrees. In still other embodiments, the medial longitudinal axis is arranged relative to the first longitudinal axis of the first portion at an angle of about 30 degrees. In yet other embodiments, the medial longitudinal axis is arranged relative to the first longitudinal axis of the first portion at an angle of about 20 degrees.

In certain embodiments of the connector hub of the present invention, the medial longitudinal axis is arranged relative to the second longitudinal axis of the second portion at an angle between about zero degrees to about 90 degrees. In other embodiments, the medial longitudinal axis is arranged relative to the second longitudinal axis of the second portion at an angle between about 10 and about 50 degrees; about 15 and about 45 degrees; and about 20 to about 40 degrees. In still other embodiments, the medial longitudinal axis is arranged relative to the second longitudinal axis of the second portion at an angle of about 30 degrees. In yet other embodiments, the medial longitudinal axis is arranged relative to the second longitudinal axis of the second portion at an angle of about 20 degrees.

In most, if not all, of the embodiments of the present invention, the connector hub serves to arrange the introducer needle at an angle relative to the syringe. Depending on the angles of each portion of the connector hub relative to other connector hub portions, the arrangement of the introducer needle relative to the syringe may be at different angles. In certain embodiments, the longitudinal axis of the introducer needle is arranged relative to the longitudinal axis of the syringe at an angle between about 10 and about 50 degrees; about 15 and about 45 degrees; and about 20 and about 40 degrees. In still other embodiments, the longitudinal axis of the introducer needle is arranged relative to the longitudinal axis of the syringe at an angle of about 30 degrees. In yet other embodiments, the longitudinal axis of the introducer needle is arranged relative to the longitudinal axis of the syringe at an angle of about 20 degrees.

In preferred embodiments, the connector hub of the present invention comprises at least one insertion port located on the medial portion. In certain embodiments, the insertion port comprises a first insertion port opening, an insertion port passage, a second insertion port opening, and an insertion port longitudinal axis. Generally, the insertion port is configured to accept a catheter guide wire or equivalent structures or instruments that facilitate installing a centerline catheter. Further, in preferred embodiments, the catheter guide wire is inserted into the insertion port via the first insertion port opening and exits the insertion port via the second insertion port opening. In still other preferred embodiments, the first insertion port opening, insertion port passage, and the second insertion port opening are in fluid communication with the medial passage.

In certain embodiments, the length of the insertion port of the connector hub may be between about 0.10 to about 0.65 inches, about 0.15 to about 0.60 inches, about 0.20 to about 0.55 inches, about 0.25 to about 0.50 inches, about 0.30 to about 0.45 inches, or about 0.35 to about 0.40 inches. In other embodiments, the length of the insertion port of the connector hub may be about 0.10, 0.15, 0.20, 0.25, 0.30, 0.35, 0.40, 0.45, 0.50, 0.55, 0.60, or 0.65 inches. In other embodiments, the insertion port of the connector hub is, e.g., about 0.55 or about 0.60 inches. In yet other embodiments, the insertion port of the connector hub is, e.g., about 0.47 inches (equating to approximately 12 mm). Other lengths are contemplated, however. Generally, the overall length of the connector hub of the present invention, including the insertion port, should be minimized.

In certain embodiments of the connector hub of the present invention, the insertion port longitudinal axis is arranged relative to the medial longitudinal axis of the medial portion at an angle between about zero degrees to about 90 degrees. In other embodiments, the insertion port longitudinal axis is arranged relative to the medial longitudinal axis of the medial portion at an angle between about 10 and about 50 degrees; about 15 and about 45 degrees; and about 20 to about 40 degrees. In still other embodiments, the insertion port longitudinal axis is arranged relative to the medial longitudinal axis of the medial portion at an angle of about 20-25 degrees. In yet other embodiments, the insertion port longitudinal axis is arranged relative to the medial longitudinal axis of the medial portion at an angle of about 30 degrees. In still other embodiments, the insertion port longitudinal axis is arranged relative to the medial longitudinal axis of the medial portion at an angle of about 20 degrees. In other embodiments, the insertion port longitudinal axis is arranged relative to the medial longitudinal axis of the medial portion at an angle of about 40 degrees.

It is also contemplated that the insertion port longitudinal axis may be arranged at an angle relative to the first longitudinal axis of the first portion and/or the second longitudinal axis of the second portion, which is in addition to the insertion port longitudinal axis being arranged at an angle relative to the medial longitudinal axis. In these latter embodiments, the angle of the insertion port longitudinal axis relative to the first and/or second longitudinal axes are about zero degrees to about 90 degrees. Further, in these latter embodiments, the insertion port longitudinal axis may extend in a radial direction different from the radial direction that the first and/or second longitudinal axes extend. That is, the insertion port longitudinal axis, the first longitudinal axis, and/or the second longitudinal axis may be arranged on the same or different planes relative to each other.

In certain embodiments of the connector hub of the present invention, the insertion port may be located on different sides (i.e., top, bottom, right, and/or left side) of the medial portion. Generally, the sides of the medial portion may be defined by reference to a radial direction relative to the surface of the patient's skin when the longitudinal axis of the introducer needle is arranged parallel to the skin surface. In the latter scenario, “top” may be at, e.g., zero (or 360) radial degrees, “right” may be at, e.g., 90 radial degrees, “bottom” may be at, e.g., 180 radial degrees, and “left” may be at, e.g., 270 radial degrees. It is contemplated, however, that the insertion port may be located within a range of radial degrees such that, moving clockwise from zero degrees, “top” may be at, e.g., about 315 to about 45 radial degrees, “right” may be at, e.g., about 45 to about 135 radial degrees, “bottom” may be at, e.g., about 135 to about 225 radial degrees, “left” may be at, e.g., about 225 to about 315 radial degrees.

The insertion port may comprise, in some embodiments, a port distal end and a port proximal end. In some embodiments, the first insertion port opening of the insertion port is located at the port proximal end and the second insertion port opening is located at the port distal end.

Further, in certain embodiments, the insertion port passage may be, e.g., a conical shape, a funnel shape, or tapered shape to facilitate guiding the tip of the catheter guide wire (e.g., the tip of a soft, flexible spring-coil guide wire) smoothly into the internal lumen of an introducer needle and onward into the subclavian vein. In this latter embodiment, the insertion port passage comprises a first port diameter and a second port diameter, wherein said second port diameter is larger than said first port diameter. In certain embodiments, the first port diameter may have a diameter of between about 0.5 mm to about 2.0 mm, with the first port diameter having a diameter of about 1 mm in some preferred embodiments. In certain embodiments, the second port diameter may have a diameter of between about 2 mm to about 5 mm, between about 2.5 mm to about 4.5 mm, with the second port diameter having a diameter of about 3 mm in some preferred embodiments. In preferred embodiments, the first port diameter is located at the port distal end (i.e., the second insertion port opening in fluid communication with the medial passage) and the second port diameter is located at the port proximal end (i.e., around the first insertion port opening). In these embodiments, the conical shape, a funnel shape, or tapered shape facilitates a smooth guide wire entry by minimizing the possibility that the guide wire will get stuck or snagged on other portions of the connector hub, and helps to aim the guide wire towards the internal lumen of the introducer needle with minimal effort needed by the physician.

In preferred embodiments, the insertion port is hermetically sealed by a seal (e.g., a hermetic seal) that may include, e.g., a membrane or a valve or equivalents. In embodiments where the insertion port contains a valve, the valve is generally a one-way valve that allows the insertion port, and thus the entire connector hub, to be hermetically sealed. Further, in embodiments comprising a valve, the valve must be configured to allow insertion of a guide wire. In embodiments where the insertion port contains a membrane, the membrane, which, in some embodiments, is made of a thin elastomeric material (e.g., a SILASTIC® silicone rubber membrane and attached to the connector hub via a medical grade SILASTIC® Type-A adhesive) is configured to hermetically seal the insertion port and thus the entire connector hub.

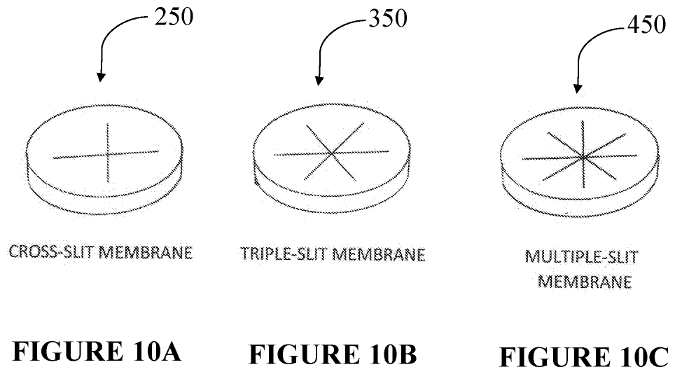

In certain embodiments, the connector hub may comprise a membrane with at least one perforation or slit, while, in other embodiments, the connector hub may comprise a membrane with a plurality of slits, e.g., at least two, at least three, or at least four perforations or slits, which maintain the ability of the membrane to provide a hermetic seal but which facilitate puncturing the membrane with the tip of, e.g., a guide wire. In certain embodiments, the membrane may comprise a single perforation or slit. In other embodiments, the perforations or slits may be in the shape of, e.g., a cross (i.e., a “+”), but other shapes of the perforation or slits are contemplated such as, e.g., the X-shaped, triple-slit and multi-slit membranes shown in, e.g., FIGS. 10A-10C.

When seeking confirmation that the introducer needle has penetrated the subclavian vein, a physician gently pulls back the plunger on the syringe to generate a slight negative pressure to aspirate venous blood. Witnessing the aspiration of venous blood confirms entry of the introducer needle into the subclavian vein and heralds the step of inserting the catheter guide wire. In certain embodiments of the present invention, the membrane comprising at least one or a plurality of perforations or slits must be strong enough to withstand the negative pressure exerted by the physician when testing for vein entry yet penetrable enough to allow easy insertion of the catheter guide wire through the membrane, into the connector hub and onward to the subclavian vein via puncturing the membrane. In this way, the membrane does not interfere with the syringe-initiated blood aspiration test.

Guide wires that may be used with the present invention include, e.g., a J-tipped guide wire sold by, e.g., Bard Medical. The J-tipped guide wire typically includes a plastic adaptor called a J-straightener that is placed on the end of the guide wire and ensures that the loop of the wire is straight prior to insertion. In preferred embodiments, the J-straightener may be used to penetrate the perforations and slits on the membrane of at least one insertion port to gain entry into the connector hub and allow the guide wire to be threaded through the connector hub into the introducer needle and onward to the subclavian vein.

In other embodiments of the connector hub of the present invention, the insertion port comprises only a first insertion port opening, wherein the first insertion port opening is arranged flush with an external surface of the medial portion. In the latter embodiment, the insertion port would not have a length extending from the external surface of the medial portion. In this way, the flush insertion port would serve as a hermetically sealed window located on an external surface of the medial portion. In the embodiments where the insertion port serves as a window on the medial portion, the insertion port may still comprise a seal (e.g., a membrane or a valve) as already described.

In certain embodiments of the connector hub of the present invention, the medial portion may comprise a plurality of insertion ports. For example, in some embodiments, the medial portion of the connector hub of the present invention may comprise at least one insertion port. In the latter embodiment, the insertion port may be located at either the top, bottom, left, or right side of the medial portion. In other embodiments, the medial portion may comprise at least two insertion ports. In the latter embodiment, an insertion port may be located on the medial portion at, e.g., the top and right sides, the top and left sides, the top and bottom sides, the left and right sides, the bottom and right sides, or the bottom and left sides. In still further embodiments, the medial portion may comprise at least three insertion ports. In the latter embodiment, an insertion port may be located on the medial portion at, e.g., the top, right, and left sides, the top, right, and bottom sides, the top, left, and bottom sides, or the bottom, right and left sides.

In preferred embodiments, connector hub of the present invention is a single unit comprising the first portion, the second portion, the medial portion, and the at least one insertion port. It is contemplated that the connector hub of the present invention may not be a single unit, however. When the connector hub of the present invention is formed as a single unit, the first opening and the first passage, the second opening and the second passage, the at least two medial openings and the medial passage, the first insertion port opening, the insertion port passage, and the second insertion port opening are all in fluid communication with each other. In certain embodiments, the foregoing passages of the connector hub of the present invention generally form a conical shape, a funnel shape, or a tapered shape to facilitate guiding the tip of the catheter guide wire (e.g., the tip of a soft, flexible spring-coil guide wire) smoothly into the internal lumen of an introducer needle and onward into the subclavian vein.

In certain embodiments, the connector hub, introducer needle, and syringe may be a single unit, but, in preferred embodiments, each of the connector hub, introducer needle, and syringe are separate components to be assembled by the physician prior to performing a centerline catheter placement.

Reference is made to FIG. 1, which is a three-dimensional depiction of a connector hub 100 according to one exemplary embodiment of the present invention. FIG. 1 depicts a connector hub 100 comprising a first portion 110 having a first opening 111, a second portion 120 having a second opening 121, a medial portion 130, and an insertion port 140 with a first insertion port opening 141. As depicted in FIG. 1, first portion 110 is offset from second portion 120, medial portion 130, and insertion port 140 at an angle such that the longitudinal axis of first portion 110 lays on a different plane from the longitudinal axes of each of second portion 120, medial portion 130, and insertion port 140. In other embodiments, it is contemplated that second portion 120 may be offset at an angle from first portion 110, medial portion 130, and insertion port 140. Connector hub 100, as shown in FIG. 1, also comprises a distal end 101 and a proximal end 102.

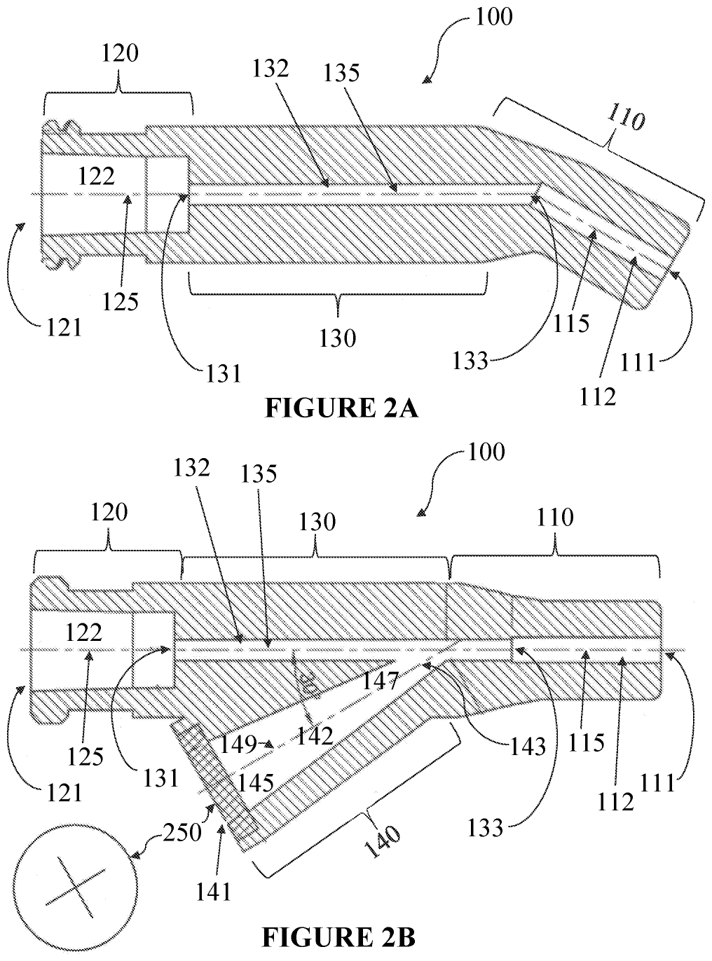

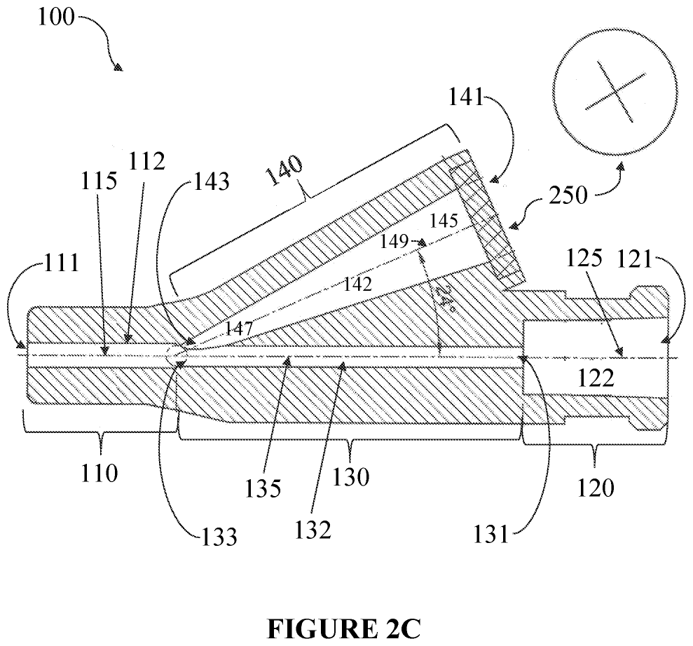

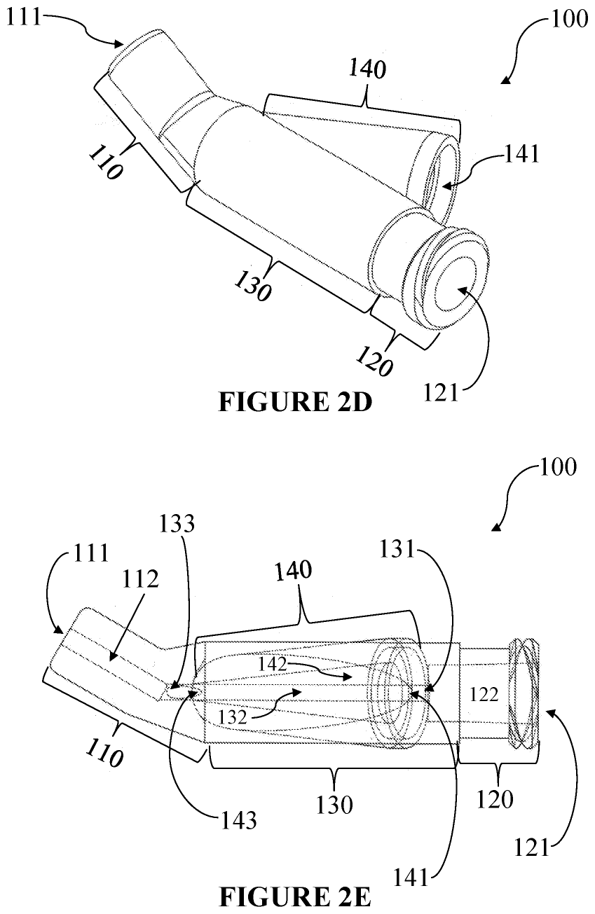

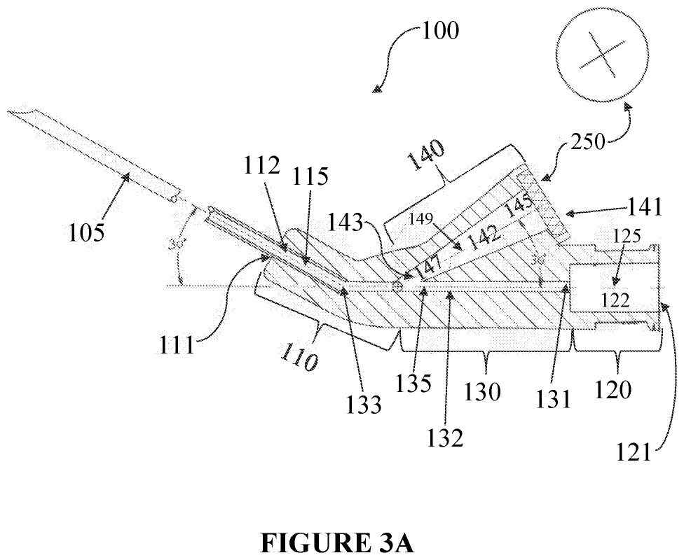

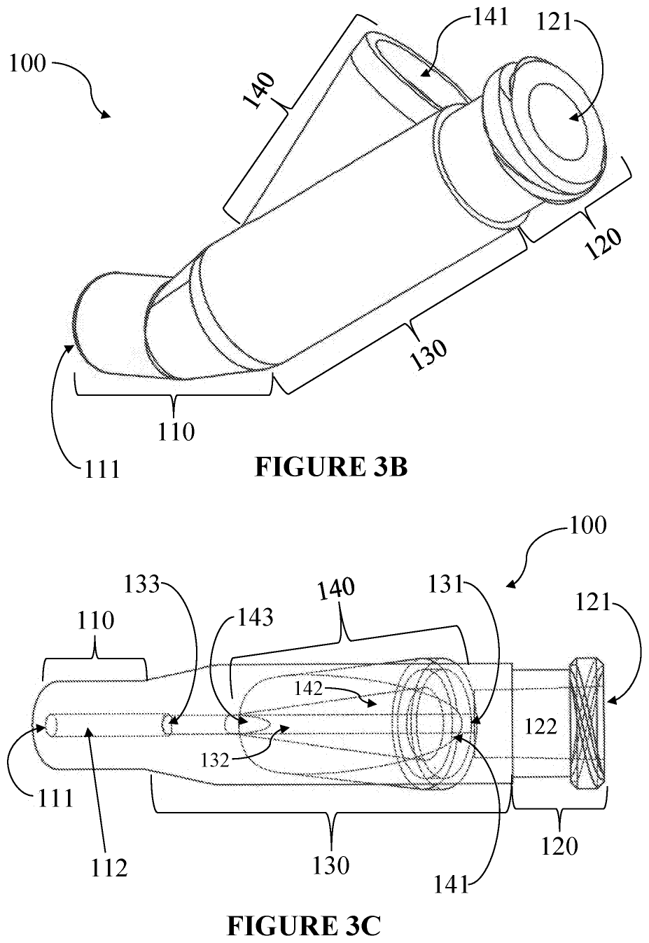

Reference is made to FIGS. 2A and 2B, which are cross-sectional depictions of a connector hub 100 according to an exemplary embodiment of the present invention. Reference is also made to FIG. 2D, which is a three-dimensional depiction of a side view of connector hub 100 depicted in FIGS. 2A and 2B, and to FIG. 2E, which is a transparent three-dimensional depiction of a side view of connector hub 100 depicted in FIGS. 2A and 2B. As shown in, e.g., FIG. 2A, an exemplary connector hub 100 of the present invention may comprise first portion 110, second portion 120, and medial portion 130. As shown in, e.g., FIG. 2B is a representative top view of connector hub 100, wherein connector hub 100 comprises first portion 110, second portion 120, medial portion 130, and insertion port 140. It is noted, however, the FIG. 2B is representative only and it is contemplated that insertion port 140 may be arranged at different locations on medial portion 130, including being located at, e.g., the top, right, left, or bottom sides of medial portion 130 or at locations considered in-between the top, right, left, or bottom sides of medial portion 130. For example, FIG. 3A is a cross-sectional depiction of connector hub 100 according to an exemplary embodiment of the present invention where insertion port 140 is located on the top of medial portion 130. Further, FIG. 3B is a three-dimensional depiction of a side view of the embodiment of connector hub 100 shown in FIG. 3A, and FIG. 3C is a transparent three-dimensional depiction of a top view of the embodiment of connector hub 100 shown in FIGS. 3A and 3B.

First portion 110 depicted in FIGS. 2A and 2B comprises first opening 111, first passage 112 and a first longitudinal axis 115. Generally, first opening 111 comprises an opening that leads into first passage 112, first passage 112 being a hollow lumen. In certain embodiments, the length of first portion 110 of connector hub 100 may be between about 0.15 to about 0.50 inches, about 0.20 to about 0.45 inches, about 0.25 to about 0.40 inches, or about 0.30 to about 0.35 inches. In other embodiments, the length of first portion 110 of connector hub 100 may be about 0.15, 0.20, 0.25, 0.30, 0.35, 0.40, 0.45, or 0.50 inches. In certain embodiments the length of first portion 110 of connector hub 100 is about 0.25 or about 0.40 inches. In the embodiment depicted in FIGS. 2A and 2B, the length of first portion 110 is, e.g., about 0.39 inches. Other lengths are contemplated, however. In certain embodiments, first portion 110 of connector hub 100 of the present invention further comprises a first distal end and a first proximal end. In some embodiments, first opening 111 of first portion 110 is located at the first distal end.

In some embodiments, first portion 110 depicted in FIGS. 2A and 2B is configured to engage with an introducer needle useful for performing a centerline catheter placement procedure. In certain embodiments, when first portion 110 is engaged with an introducer needle, the first longitudinal axis 115 of first portion 110 is arranged relative to a longitudinal axis of the introducer needle at an angle of about zero degrees, although other angles are contemplated. In some embodiments, first portion 110 of connector hub 100 of the present invention may comprise a male luer fitting that is configured to engage with a female luer fitting on an introducer needle. For example, first opening 111 at the first distal end of the first portion 110 may be formed as a male luer fitting. In other embodiments, however, an introducer needle may be integrally formed with, and/or embedded within, first portion 110 of connector hub 100 of the present invention. In all contemplated embodiments, when first portion 110 of connector hub 100 is engaged with the introducer needle, the introducer needle's internal lumen and first passage 112 are fluidly connected.

Second portion 120 depicted in FIGS. 2A and 2B comprises second opening 121, second passage 122 and a second longitudinal axis 125. Generally, second opening 121 includes an opening that leads into second passage 122, second passage 122 being a hollow lumen. In certain embodiments, the length of second portion 120 of connector hub 100 may be between about 0.10 to about 0.40 inches, about 0.15 to about 0.35 inches, or about 0.20 to about 0.30 inches. In other embodiments, the length of second portion 120 of connector hub 100 may be about 0.10, 0.15, 0.20, 0.25, 0.30, 0.35, or 0.40 inches. In the embodiment depicted in FIGS. 2A and 2B, the length of second portion 120 of connector hub 100 is, e.g., about 0.30 inches. Other lengths are contemplated, however. In certain embodiments, second portion 120 of connector hub 100 of the present invention further comprises a second distal end and a second proximal end. In some embodiments, second opening 121 of second portion 120 is located at the second proximal end.

In some embodiments, second portion 120 is configured to engage with a syringe useful for performing a centerline catheter placement procedure. In certain embodiments, when second portion 120 is engaged with a syringe, the second longitudinal axis 125 of second portion 120 is arranged relative to a longitudinal axis of the syringe at an angle of about zero degrees, although other angles are contemplated. In certain embodiments, second portion 120 of connector hub 100 of the present invention may comprise a female luer fitting that is configured to engage with a male luer fitting on a syringe. In other embodiments, however, a syringe may be integrally formed with, and/or embedded within, second portion 120 of connector hub 100 of the present invention. In all contemplated embodiments, when second portion 120 of connector hub 100 is engaged with the syringe, the syringe's hollow receptacle barrel and second passage 122 are fluidly connected.

In certain embodiments, the first distal end of first portion 110 is the same as distal end 101 (see FIG. 1) of connector hub 100 of the present invention. Further, in certain embodiments, the second proximal end of second portion 120 is the same as proximal end 102 (see FIG. 1) of connector hub 100 of the present invention.

Medial portion 130 depicted in FIGS. 2A and 2B is arranged between first portion 110 and second portion 120, wherein medial portion 130 comprises at least two medial openings 131 and 133, medial passage 132, and a medial longitudinal axis 135. Generally, medial passage 132 is a hollow lumen that is fluidly connected with both first passage 112 of first portion 110 as well as second passage 122 of second portion 120. Medial portion 130 comprises a medial distal end and a medial proximal end. In certain embodiments of connector hub 100 of the present invention, and as depicted in, e.g., FIGS. 2A and 2B, first portion 110 is located at the medial distal end of medial portion 130. In certain embodiments of connector hub 100 of the present invention, and as depicted in, e.g., FIGS. 2A and 2B, second portion 120 is located at the medial proximal end of medial portion 130.

In certain embodiments of connector hub 100 of the present invention, the medial longitudinal axis 135 is arranged relative to the first longitudinal axis 115 of first portion 110 at an angle between about zero degrees and about 90 degrees. In other embodiments, the medial longitudinal axis 135 is arranged relative to the first longitudinal axis 115 of first portion 110 at an angle between about 10 and about 50 degrees; about 15 and about 45 degrees; and about 20 and about 40 degrees. In still other embodiments, and as depicted in, e.g., FIGS. 2A and 2B, the medial longitudinal axis 135 is arranged relative to the first longitudinal axis 115 of first portion 110 at an angle of about 30 degrees.

In certain embodiments of connector hub 100 of the present invention, the medial longitudinal axis 135 is arranged relative to the second longitudinal axis 125 of second portion 120 at an angle between about zero degrees to about 90 degrees. In other embodiments, the medial longitudinal axis 135 is arranged relative to the second longitudinal axis 125 of second portion 120 at an angle between about 10 and about 50 degrees; about 15 and about 45 degrees; and about 20 to about 40 degrees. In still other embodiments, the medial longitudinal axis 135 is arranged relative to the second longitudinal axis 125 of second portion 120 at an angle of about 30 degrees. In the embodiment of connector hub 100 depicted in FIGS. 2A and 2B, the medial longitudinal axis 135 is arranged relative to the second longitudinal axis 125 of second portion 120 at an angle of zero degrees.

Insertion port 140 depicted in FIG. 2B comprises first insertion port opening 141, insertion port passage 142, second insertion port opening 143, and an insertion port longitudinal axis 149. Generally, insertion port 140 is configured to accept a catheter guide wire such as, e.g., a guide wire or equivalent structures or instruments that facilitate installing a centerline catheter. Further, in preferred embodiments, the catheter guide wire is inserted into insertion port 140 via first insertion port opening 141 and exits insertion port 140 via second insertion port opening 143. In still other preferred embodiments, first insertion port opening 141, insertion port passage 142, and second insertion port opening 143 are in fluid communication with medial passage 132.

In certain embodiments, the length of insertion port 140 of connector hub 100 may be between about 0.10 to about 0.65 inches, about 0.15 to about 0.60 inches, about 0.20 to about 0.55 inches, about 0.25 to about 0.50 inches, about 0.30 to about 0.45 inches, or about 0.35 to about 0.40 inches. In other embodiments, the length of insertion port 140 of connector hub 100 may be about 0.10, 0.15, 0.20, 0.25, 0.30, 0.35, 0.40, 0.45, 0.50, 0.55, 0.60, or 0.65 inches. In other embodiments, such as the representative embodiment depicted FIG. 2B, insertion port 140 of connector hub 100 is, e.g., about 0.55 to about 0.60 inches, or about 0.575 inches. Other lengths are contemplated, however.

In certain embodiments of connector hub 100 of the present invention, the insertion port longitudinal axis 149 is arranged relative to the medial longitudinal axis 135 of medial portion 130 at an angle between about zero degrees to about 90 degrees. In other embodiments, the insertion port longitudinal axis 149 is arranged relative to the medial longitudinal axis 135 of medial portion 130 at an angle between about 10 and about 50 degrees; about 15 and about 45 degrees; and about 20 to about 40 degrees. In still other embodiments, such as the representative embodiment depicted in FIG. 2C, the insertion port longitudinal axis 149 is arranged relative to the medial longitudinal axis 135 of medial portion 130 at an angle of about 24 degrees. In yet other embodiments, such as the representative embodiment depicted in FIG. 2B, the insertion port longitudinal axis 149 is arranged relative to the medial longitudinal axis 135 of medial portion 130 at an angle of about 30 degrees.

It is also contemplated that the insertion port longitudinal axis 149 may be arranged at an angle relative to the first longitudinal axis 115 of first portion 110 and/or the second longitudinal axis 125 of second portion 120, which is in addition to the insertion port longitudinal axis 149 being arranged at an angle relative to the medial longitudinal axis 135. In these latter embodiments, the angle of the insertion port longitudinal axis 149 relative to the first and/or second longitudinal axes are about zero degrees to about 90 degrees, about 10 degrees to about 80 degrees, about 15 degrees to about 75 degrees, about 20 degrees to about 70 degrees, about 25 degrees to about 65 degrees, about 30 degrees to about 60 degrees, about 35 degrees to about 55 degrees, and about 40 degrees to about 50 degrees.

Further, in these latter embodiments, the insertion port longitudinal axis 149 may extend in a radial direction different from the radial direction that the first and/or second longitudinal axes extend. That is, the insertion port longitudinal axis 149, the first longitudinal axis 115, and/or the second longitudinal axis 125 may be arranged on the same or different planes relative to each other. A representative example of such an embodiment is depicted in FIG. 2A—a side view of connector hub 100 of the present invention depicting first portion 110 arranged at an angle relative medial portion 130—and FIG. 2B—a top view of connector hub 100 of the present invention depicting insertion port 140 arranged at an angle relative to medial portion 130. With respect to the embodiment of connector hub 100 depicted in FIGS. 2A and 2B, the insertion port longitudinal axis 149 of insertion port 140 is arranged at an angle relative to the first longitudinal axis 115 of first portion 110, and the insertion port longitudinal axis 149 extends in a radial direction different than the first longitudinal axis 115 of first portion 110.

The representative embodiment of connector hub 100 depicted in FIG. 2B shows insertion port 140 arranged on, e.g., a right side of medial portion 130 when viewed from the proximal end. But other arrangements are contemplated. For example, in certain embodiments, insertion port 140 may be located on different sides (i.e., top, bottom, right, and/or left side) of medial portion 130. Generally, the sides of medial portion 130 may be defined by reference to a radial direction relative to the surface of the patient's skin when the longitudinal axis of the introducer needle is arranged parallel to the skin surface. In the latter scenario, “top” may be at, e.g., zero (or 360) radial degrees, “right” may be at, e.g., 90 radial degrees, “bottom” may be at, e.g., 180 radial degrees, and “left” may be at, e.g., 270 radial degrees. It is contemplated, however, that insertion port 140 may be located within a range of radial degrees such that, moving clockwise from zero degrees, “top” may be at, e.g., about 315 to about 45 radial degrees, “right” may be at, e.g., about 45 to about 135 radial degrees, “bottom” may be at, e.g., about 135 to about 225 radial degrees, “left” may be at, e.g., about 225 to about 315 radial degrees.

The representative example of insertion port 140 depicted in FIG. 2B also comprises a port distal end 147 and a port proximal end 145. In the embodiment depicted in FIG. 2B, first insertion port opening 141 of insertion port 140 is located at port proximal end 145 and second insertion port opening 143 is located at port distal end 147.

Further, in the embodiment depicted in FIG. 2B, insertion port passage 142 may be, e.g., a conical shape, a funnel shape, or tapered shape to facilitate guiding the tip of the catheter guide wire smoothly into medial passage 132 towards the internal lumen of an introducer needle and onward into the subclavian vein. In the embodiment depicted in FIG. 2B, insertion port passage 142 comprises a first port diameter and a second port diameter, wherein said second port diameter is larger than said first port diameter. In preferred embodiments, the first port diameter is located at port distal end 147 (i.e., second insertion port opening 143 in fluid communication with medial passage 132) and the second port diameter is located at port proximal end 145 (i.e., around first insertion port opening 141).

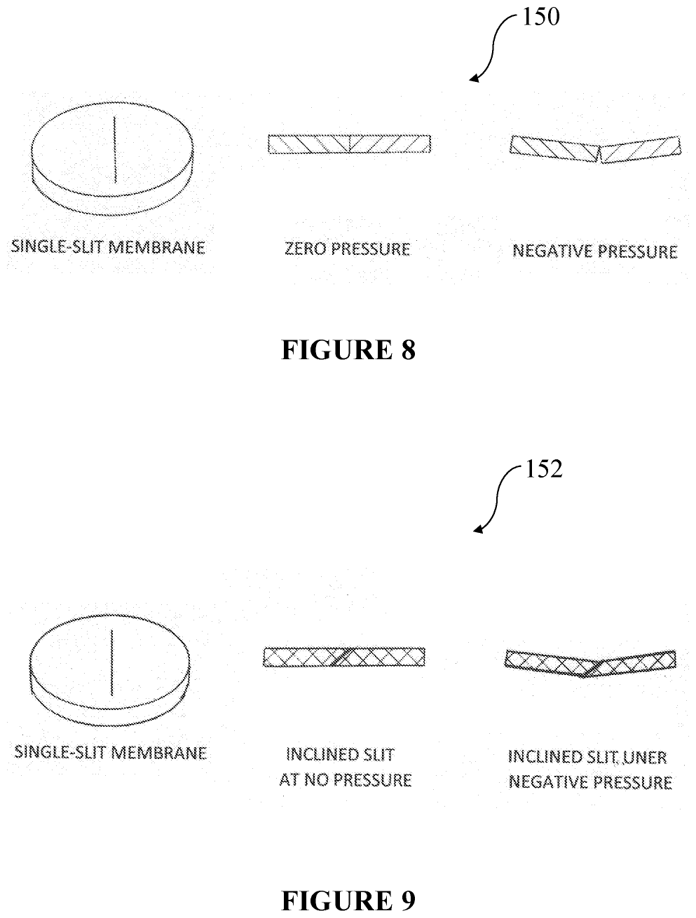

In preferred embodiments, insertion port 140 is hermetically sealed by a seal that may include, e.g., a membrane or a valve or equivalents. In the embodiment depicted in FIG. 2B, insertion port 140 is hermetically sealed by membrane 250, which may be made of, e.g., a SILASTIC® silicone rubber membrane and attached to the connector hub via a medical grade SILASTIC® Type-A adhesive. In certain embodiments, membrane 250 may comprise perforations or slits as shown in FIG. 2B, which maintain the ability of membrane 250 to provide a hermetic seal but which facilitate puncturing membrane 250 with the tip of, e.g., a guide wire. In the embodiments depicted in FIG. 2B, the perforations or slits on membrane 250 are in the shape of, e.g., a cross (i.e., a “+”). Other shapes of the perforation or slits are contemplated, however. For example, membrane 250 may have a single perforation or slit, such as depicted in, e.g., FIGS. 8 and 9.

Reference is made to FIG. 3A, which is a cross-sectional depiction of connector hub 100 according to certain aspects of the present invention, FIG. 3B, which is a three-dimensional depiction of a side view of connector hub 100 shown in FIG. 3A, and FIG. 3C, which is a transparent three-dimensional depiction of a top view of connector hub 100 shown in FIG. 3A. Generally, the embodiment of connector hub 100 depicted in FIGS. 3A-3C is similar to the embodiment of connector hub 100 depicted in FIGS. 2A, 2B, 2C, 2D, and 2E, except that insertion port 140 is located on the top of medial portion 130 of the connector hub 100 depicted in FIGS. 3A-3C. Further, an introducer needle 105 is depicted in FIG. 3A as embedded in first portion 110 of connector hub 100. But other embodiments involving introducer needle 105 are also contemplated such as, e.g., insertion needle 105 engaging with first portion 110 via a luer lock connection (i.e., introducer needle 105 comprising a male luer fitting configured to engage with a female luer fitting on first portion 110).

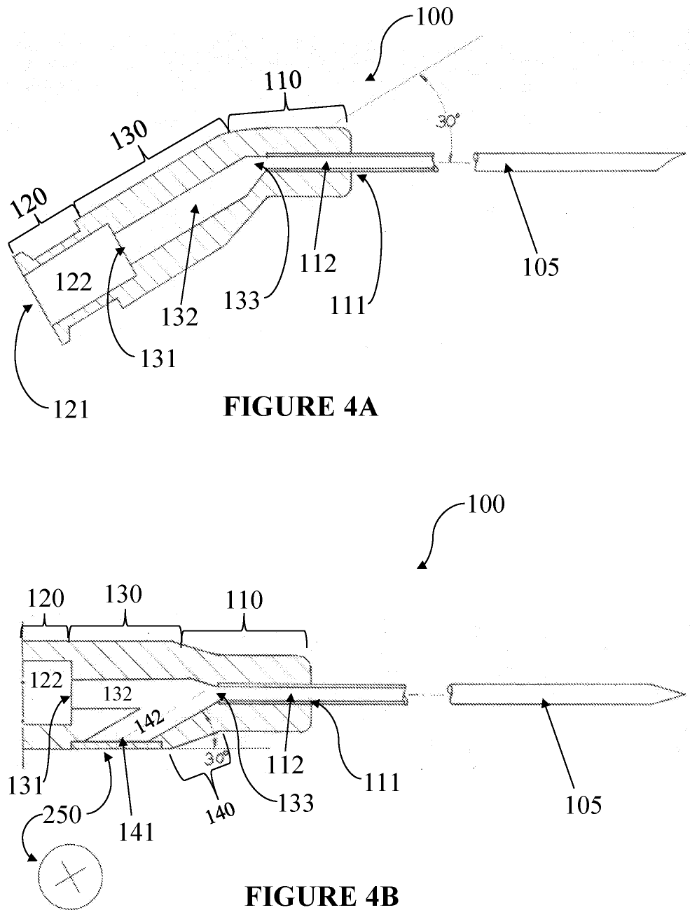

Reference is now made to FIGS. 4A and 4B, which are cross-sectional depictions of a side view and a top view, respectively, of another embodiment of connector hub 100 according to certain aspects of the present invention. Generally, the embodiment of connector hub 100 depicted in FIGS. 4A and 4B is similar to the embodiment of connector hub 100 depicted in FIGS. 2A, 2B, 2C, 2D, 2E, and/or FIGS. 3A-3C, except that first insertion port opening 141 is arranged flush with an external surface of medial portion 130 (see FIG. 4B). Further, an introducer needle 105 is depicted in FIGS. 4A and 4B as embedded in first portion 110 of connector hub 100. But other embodiments involving introducer needle 105 are also contemplated such as, e.g., insertion needle 105 engaging with first portion 110 via a luer lock connection (i.e., introducer needle 105 comprising a male luer fitting configured to engage with a female luer fitting on first portion 110).

As shown in the embodiment of connector hub 100 depicted in FIG. 4A, the medial longitudinal axis 135 of medial portion 130 is arranged relative to the first longitudinal axis 115 of first portion 110 at an angle of about 30 degrees, although other angles are contemplated. Further, as shown in the embodiment of connector hub 100 depicted in FIG. 4B, the insertion port longitudinal axis 149 of insertion port 140 is arranged relative to the medial longitudinal axis 135 of medial portion 130—which is parallel to external surface of medial portion 130 that first insertion port opening 141 is flush with (FIG. 4B)—at an angle of about 30 degrees, although other angles are contemplated.

In certain embodiments, the longitudinal axis of insertion port 140 is arranged relative to the first longitudinal axis 115 of first portion 110—which, in most embodiments, is equivalent to the longitudinal axis of introducer needle 105—at an angle of about 25 degrees to about 55 degrees, about 30 degrees to about 50 degrees, or about 35 degrees to about 45 degrees. In certain preferred embodiments, the longitudinal axis of insertion port 140 is arranged relative to the first longitudinal axis 115 of first portion 110—and thus the longitudinal axis of introducer needle 105—at an angle of about 40 degrees. In general, however, it is contemplated that the angle of the longitudinal axis of insertion port 140 relative to the longitudinal axis of introducer needle 105 is as minimal as possible, as the more linear the path between the insertion port and the introducer needle the easier it become to advance the guide wire with minimal resistance.

The representative embodiment of connector hub 100 depicted in FIG. 4B shows insertion port 140 arranged on, e.g., a right side of medial portion 130. But, similar to the embodiment depicted in FIG. 2B, other arrangements are contemplated. For example, in certain embodiments, insertion port 140 may be located on different sides (i.e., top, bottom, right, and/or left side) of medial portion 130. FIGS. 3A-3C, e.g., depict an embodiment of connection hub 100 with insertion port 140 located on top of medial portion 130. Further, in the embodiment depicted in FIG. 4B, insertion port passage 142 may be, e.g., a conical shape, a funnel shape, or tapered shape to facilitate guiding the tip of the catheter guide wire (e.g., the tip of a soft, flexible spring-coil guide wire) smoothly into medial passage 132 towards the internal lumen of introducer needle 105 and onward into the subclavian vein. The representative embodiment of connector hub 100 depicted in FIG. 4B also shows a seal (e.g., membrane 250) as described with reference to FIG. 2B (see also FIGS. 8, 9, and 10A-10C).

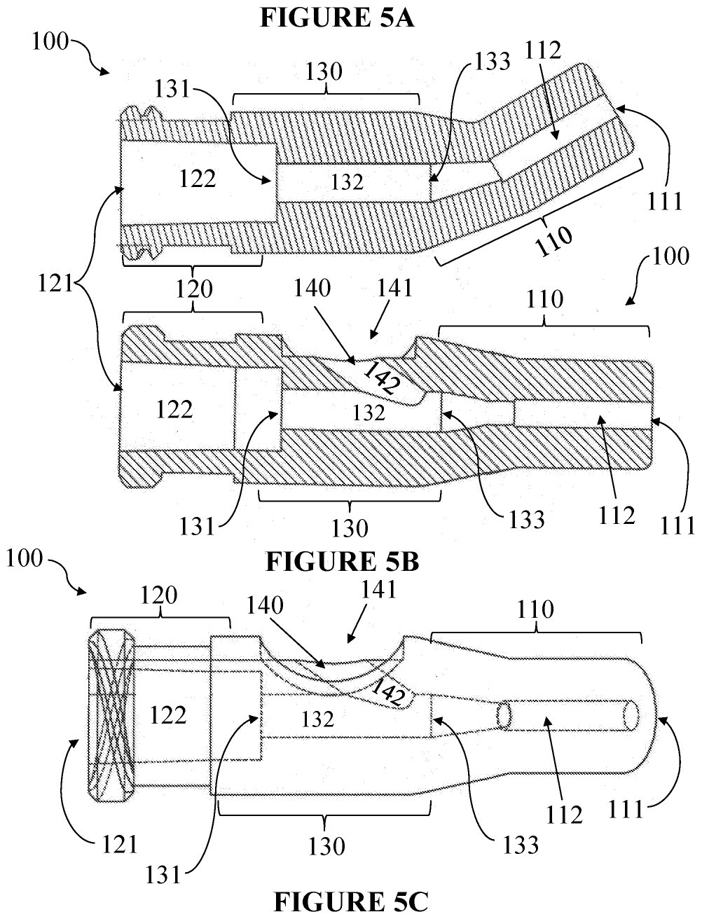

Reference is made to FIGS. 5A and 5B, which are cross-sectional depictions of a side view and a top view of connector hub 100 according to certain aspects of the present invention. Reference is also made to FIG. 5C, which is a transparent three-dimensional depiction of connector hub 100 shown in FIGS. 5A and 5B according to certain aspects of the present invention. Generally, the embodiment of connector hub 100 shown in FIGS. 5A-5C includes the same components as the embodiments of connector hub 100 shown in, e.g., FIGS. 1, 2A-2B, 3A-3C, and 4A-4B but with some notable differences. For example, as shown in, e.g., FIGS. 5A-5C, a portion of first passage 112 adjacent to medial opening 133 is a conical shape, a funnel shape, or tapered shape to facilitate guiding the tip of the catheter guide wire (e.g., the tip of a soft, flexible spring-coil guide wire) smoothly into the internal lumen of an introducer needle and onward into the subclavian vein. Similarly, insertion port passage 142, as shown in, e.g., FIGS. 5A-5C, is also a conical shape, a funnel shape, or tapered shape to facilitate guiding the tip of the catheter guide wire (e.g., the tip of a soft, flexible spring-coil guide wire) smoothly into medial passage 132, first passage 112, the internal lumen of an introducer needle, and onward into the subclavian vein. Additionally, as shown in, e.g., FIG. 5C, first insertion port opening 141 has a concave shape, which assists in easing the angle at which the physician must insert the catheter guide wire through first insertion port opening 141 and into insertion port passage 142. Although not shown in FIGS. 5A-5C, it is contemplated that the embodiment of connector hub 100 shown in those figures may also include a seal such as, e.g., a membrane 250 as shown in, e.g., FIGS. 2B, 3A, and 4B (see also FIGS. 8, 9, and 10A-10C).

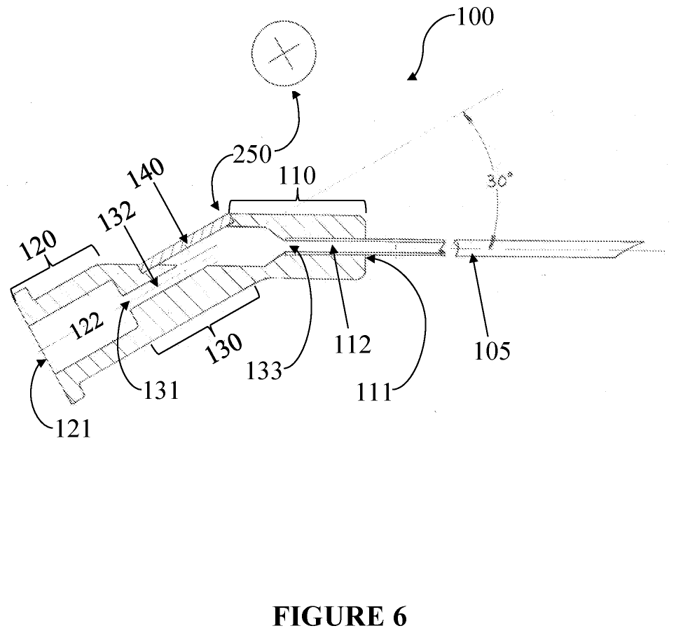

Reference is made to FIG. 6, which is a cross-sectional depiction of a side view of connector hub 100 according to certain aspects of the present invention. Generally, the embodiment of connector hub 100 shown in FIG. 6 includes the same components as the embodiments of connector hub 100 shown in, e.g., FIGS. 1, 2A-2B, 3A-3C, 4A-4B, and 5A-5C but with some notable differences. For example, the embodiment of connector hub 100 shown in FIG. 6 still has an insertion port 140 but lacks certain components associated with insertion port 140 such as, e.g., insertion port passage 142. As another example, in the embodiment of connector hub 100 shown in FIG. 6, insertion port 140 and first insertion port opening 141 are substantially the same component, as insertion port 140—as shown in FIG. 6—is just an opening on the external surface of medial portion 130 hermetically sealed by a seal such as, e.g., membrane 250.

Further, the embodiment of connector hub 100 shown in FIG. 6 depicts insertion port 140 on the bottom of medial portion 130, but it is contemplated that insertion port 140 may be arranged at different locations on medial portion 130, including being located at, e.g., the top, right, or left sides of medial portion 130 or at locations considered in-between the top, right, left, or bottom sides of medial portion 130.

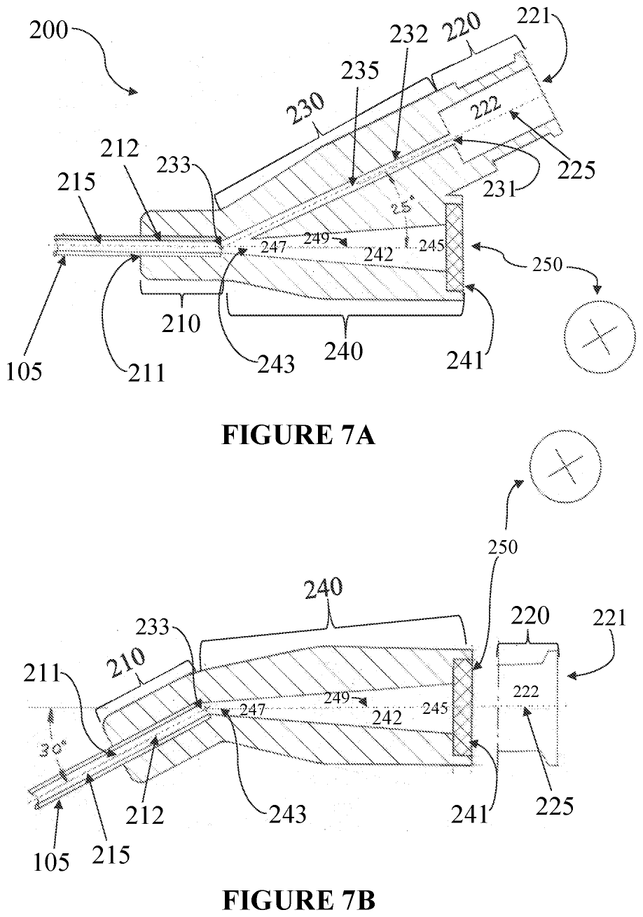

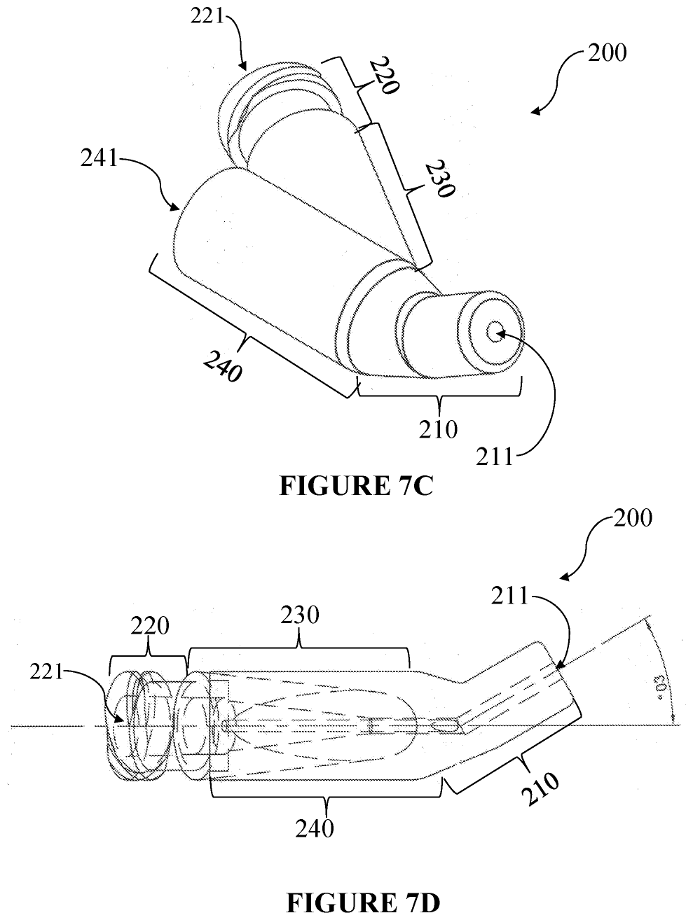

Reference is made to FIGS. 7A and 7B, which are cross-sectional depictions of a top view and a side view, respectively, of connector hub 200 according to certain aspects of the present invention. Reference is also made to FIG. 7C, which is a three-dimensional depiction of connector hub 200 depicted in FIGS. 7A and 7B, and FIG. 7D, which is a transparent three-dimensional depiction of connector hub 200 depicted in FIGS. 7A and 7B. FIG. 7A is a top view of connector hub 200 comprising a first portion 210 having a first opening 211, a second portion 220 having a second opening 221, a medial portion 230, and an insertion port 240 with a first insertion port opening 241. As depicted in FIGS. 7A and 7B, first portion 210 is offset from second portion 220, medial portion 230, and insertion port 240 at an angle such that the longitudinal axis of the first portion 210 lays on a different plane from the longitudinal axes of each of second portion 220, medial portion 230, and insertion port 240 (see, e.g., FIG. 7C).

First portion 210 depicted in FIGS. 7A and 7B comprises first opening 211, first passage 212 and a first longitudinal axis 215. Generally, first opening 211 comprises an opening that leads into first passage 212, first passage 212 being a hollow lumen. In certain embodiments, the length of first portion 210 of connector hub 200 may be between about 0.15 to about 0.50 inches, about 0.20 to about 0.45 inches, about 0.25 to about 0.40 inches, or about 0.30 to about 0.35 inches. In other embodiments, the length of first portion 210 of connector hub 200 may be about 0.15, 0.20, 0.25, 0.30, 0.35, 0.40, 0.45, or 0.50 inches. In certain embodiments the length of first portion 210 of connector hub 200 is about 0.25 or about 0.40 inches. In the embodiment depicted in FIGS. 7A and 7B, the length of first portion 210 is, e.g., about 0.24 inches. Other lengths are contemplated, however. In certain embodiments, first portion 210 of connector hub 200 of the present invention further comprises a first distal end and a first proximal end. In some embodiments, first opening 211 of first portion 210 is located at the first distal end.

In some embodiments, first portion 210 depicted in FIGS. 7A and 7B is configured to engage with introducer needle 105 useful for performing a centerline placement procedure. In certain embodiments, when first portion 210 is engaged with introducer needle 105, the first longitudinal axis 215 of first portion 210 is arranged relative to a longitudinal axis of introducer needle 105 at an angle of about zero degrees, although other angles are contemplated. In some embodiments, first portion 210 of connector hub 200 of the present invention may comprise a male luer fitting that is configured to engage with a female luer fitting on introducer needle 105. For example, first opening 211 at the first distal end of the first portion 210 may be formed as a male luer fitting. In other embodiments, however, introducer needle 105 may be integrally formed with, and/or embedded within, first portion 210 of connector hub 200 of the present invention. In all contemplated embodiments, when first portion 210 of connector hub 200 is engaged with the introducer needle, the introducer needle's internal lumen and first passage 212 are fluidly connected.

Second portion 220 depicted in FIGS. 7A and 7B comprises second opening 221, second passage 222 and a second longitudinal axis 225. Generally, second opening 221 includes an opening that leads into second passage 222, second passage 222 being a hollow lumen. In certain embodiments, the length of second portion 220 of connector hub 200 may be between about 0.10 to about 0.40 inches, about 0.15 to about 0.35 inches, or about 0.20 to about 0.30 inches. In other embodiments, the length of second portion 220 of connector hub 200 may be about 0.10, about 0.15, about 0.20, about 0.25, about 0.30, about 0.35, or about 0.40 inches. In the embodiment depicted in FIGS. 7A and 7B, the length of second portion 220 of connector hub 200 is, e.g., about 0.30 inches. Other lengths are contemplated, however. In certain embodiments, second portion 220 of connector hub 200 of the present invention further comprises a second distal end and a second proximal end. In some embodiments, second opening 221 of second portion 220 is located at the second proximal end.

In some embodiments, second portion 220 is configured to engage with a syringe useful for performing a centerline placement procedure. In certain embodiments, when second portion 220 is engaged with a syringe, the second longitudinal axis 225 of second portion 220 is arranged relative to a longitudinal axis of the syringe at an angle of about zero degrees, although other angles are contemplated. In certain embodiments, second portion 220 of connector hub 200 of the present invention may comprise a female luer fitting that is configured to engage with a male luer fitting on a syringe. In other embodiments, however, a syringe may be integrally formed with, and/or embedded within, second portion 220 of connector hub 200 of the present invention. In all contemplated embodiments, when second portion 220 of connector hub 200 is engaged with the syringe, the syringe's hollow receptacle barrel and second passage 222 are fluidly connected.

Medial portion 230 depicted in FIG. 7A, but hidden in FIG. 7B, is arranged between first portion 210 and second portion 220, wherein medial portion 230 comprises at least two medial openings 231 and 233, medial passage 232, and a medial longitudinal axis 235. Generally, medial passage 232 is a hollow lumen that is fluidly connected with both first passage 212 of first portion 210 as well as second passage 222 of second portion 220. Medial portion 230 comprises a medial distal end and a medial proximal end. In certain embodiments of connector hub 200 of the present invention, and as depicted in, e.g., FIGS. 7A and 7B, first portion 210 is located at the medial distal end of medial portion 230. In certain embodiments of connector hub 200 of the present invention, and as depicted in, e.g., FIGS. 7A and 7B, second portion 220 is located at the medial proximal end of medial portion 230.

In certain embodiments of connector hub 200 of the present invention, the medial longitudinal axis 235 is arranged relative to the first longitudinal axis 215 of first portion 210 at an angle between about zero degrees and about 90 degrees. In other embodiments, the medial longitudinal axis 235 is arranged relative to the first longitudinal axis 215 of first portion 210 at an angle between about 20 and about 40 degrees, for example, from about 10-50 degrees or from about 15-45 degrees. In still other embodiments, and as depicted in, e.g., FIGS. 7A and 7B, the medial longitudinal axis 235 is arranged relative to the first longitudinal axis 215 of first portion 210 at an angle between about 20 and about 30 degrees.

In certain embodiments of connector hub 200 of the present invention, the medial longitudinal axis 235 is arranged relative to the second longitudinal axis 225 of second portion 220 at an angle between about zero degrees to about 90 degrees. In other embodiments, the medial longitudinal axis 235 is arranged relative to the second longitudinal axis 225 of second portion 220 at an angle between about 10 and about 50 degrees; about 15 and about 45 degrees; and about 20 to about 40 degrees. In still other embodiments, the medial longitudinal axis 235 is arranged relative to the second longitudinal axis 225 of second portion 220 at an angle of about 30 degrees. In the embodiment of connector hub 200 depicted in FIGS. 7A and 7B, the medial longitudinal axis 235 is arranged relative to the second longitudinal axis 225 of second portion 220 at an angle of zero degrees.

Insertion port 240 depicted in FIGS. 7A and 7B comprises first insertion port opening 241, insertion port passage 242, second insertion port opening 243, and an insertion port longitudinal axis 249. Generally, insertion port 240 is configured to accept a catheter guide wire or equivalent structures that facilitate installing a centerline catheter. Further, in preferred embodiments, the catheter guide wire is inserted into insertion port 240 via first insertion port opening 241 and exits insertion port 240 via second insertion port opening 243. In still other preferred embodiments, first insertion port opening 241, insertion port passage 242, and second insertion port opening 243 are in fluid communication with medial passage 232.

In certain embodiments, the length of insertion port 240 of connector hub 200 may be between about 0.10 to about 0.70 inches, about 0.15 to about 0.65 inches, about 0.20 to about 0.60 inches, about 0.25 to about 0.55 inches, about 0.30 to about 0.50 inches, about 0.35 to about 0.45 inches, or about 0.40 inches. In other embodiments, the length of insertion port 240 of connector hub 200 may be about 0.10, 0.15, 0.20, 0.25, 0.30, 0.35, 0.40, 0.45, 0.50, 0.55, 0.60, 0.65, or 0.70 inches. In other embodiments, such as the representative embodiment depicted FIGS. 7A and 7B, insertion port 240 of connector hub 200 is, e.g., about 0.65 to about 0.70 inches, or about 0.69 inches. Other lengths are contemplated, however.

In certain embodiments of connector hub 200 of the present invention, the insertion port longitudinal axis 249 is arranged relative to the medial longitudinal axis 235 of medial portion 230 at an angle between about zero degrees to about 90 degrees. In other embodiments, the insertion port longitudinal axis 249 is arranged relative to the medial longitudinal axis 235 of medial portion 230 at an angle between about 10 and about 50 degrees; about 15 and about 45 degrees; and about 20 to about 40 degrees. In still other embodiments, such as the representative embodiment depicted in FIG. 7A, the insertion port longitudinal axis 249 is arranged relative to the medial longitudinal axis 235 of medial portion 230 at an angle of about 25 degrees. In yet other embodiments, the insertion port longitudinal axis 249 is arranged relative to the medial longitudinal axis 235 of medial portion 230 at an angle of about 30 degrees.

It is also contemplated that the insertion port longitudinal axis 249 may be arranged at an angle relative to the first longitudinal axis 215 of first portion 210 and/or the second longitudinal axis 225 of second portion 220, which is in addition to the insertion port longitudinal axis 249 being arranged at an angle relative to the medial longitudinal axis 235. In these latter embodiments, the angle of the insertion port longitudinal axis 249 relative to the first and/or second longitudinal axes 215/225 are about zero degrees to about 90 degrees. Further, in these latter embodiments, the insertion port longitudinal axis 249 may extend in a radial direction different from the radial direction that the first and/or second longitudinal axes 215/225 extend. That is, the insertion port longitudinal axis 249, the first longitudinal axis 215, and/or the second longitudinal axis 225 may be arranged on the same or different planes relative to each other. A representative example of such an embodiment is depicted in FIG. 7B—a side view of connector hub 200 of the present invention—which depicts first portion 210 arranged at an angle relative to insertion port 230—and FIG. 7A—a top view of connector hub 200 of the present invention—which depicts insertion port 240 arranged at an angle relative to medial portion 230. With respect to the embodiment of connector hub 200 depicted in FIGS. 7A and 7B, the insertion port longitudinal axis 249 of insertion port 240 is arranged at an angle relative to the first longitudinal axis 215 of first portion 210, and the insertion port longitudinal axis 249 extends in a radial direction different than the first longitudinal axis 215 of first portion 210.

As depicted in FIGS. 7A and 7B, medial portion 230 (and the connected second portion 220) may be considered as a side branch to insertion port 240 as opposed to, e.g., insertion port 140 being considered as a side branch to medial portion 130 in, e.g., FIGS. 2A and 2B. The latter observation is one of the key differences between the representative embodiments of connector hub 100 depicted in FIGS. 1, 2A-2E, 3A-3C, 4A-4B, and 5A-5C and the representative embodiments of connector hub 200 depicted in FIGS. 7A-7D.

The representative embodiment of connector hub 200 depicted in FIGS. 7A-7D shows insertion port 240 arranged on, e.g., a right side of medial portion 230. But other arrangements are contemplated. For example, in certain embodiments, insertion port 240 may be located on different sides (i.e., top, bottom, right, and/or left side) of medial portion 230. In another interpretation of the representative embodiment of connector hub 200 depicted in FIGS. 7A-7D, medial portion 230 may be located on different sides (i.e., top bottom, right, and/or left) of insertion port 240.

The representative example of insertion port 240 depicted in FIGS. 7A-7D also comprises a port distal end 247 and a port proximal end 245. In the embodiment depicted in FIGS. 7A-7D, first insertion port opening 241 of insertion port 240 is located at port proximal end 245 and second insertion port opening 243 is located at port distal end 247.

Further, in the embodiment depicted in FIGS. 7A and 7B as well as FIG. 7D, insertion port passage 242 may be, e.g., a conical shape, a funnel shape, or tapered shape to facilitate guiding the tip of the catheter guide wire (e.g., the tip of a soft, flexible spring-coil guide wire) smoothly into medial passage 232 (via second insertion port opening 243) towards the internal lumen of introducer needle 105 and onward into the subclavian vein. In the embodiment depicted in FIGS. 7A and 7B, insertion port passage 242 comprises a first port diameter and a second port diameter, wherein the second port diameter is larger than the first port diameter. In preferred embodiments, the first port diameter is located at port distal end 247 (i.e., second insertion port opening 243 in fluid communication with medial passage 232) and the second port diameter is located at port proximal end 245 (i.e., around first insertion port opening 241).

In preferred embodiments, insertion port 240 is hermetically sealed by a seal that may include, e.g., a membrane or a valve or equivalents. In the embodiment depicted in FIGS. 7A and 7B, insertion port 240 is hermetically sealed by membrane 250, which may be made of, e.g., a SILASTIC® silicone rubber membrane & Type-A adhesive. In certain embodiments, membrane 250 may comprise perforations or slits as shown in FIGS. 7A and 7B, which maintain the ability of membrane 250 to provide a hermetic seal but which facilitate puncturing membrane 250 with the tip of, e.g., a guide wire. The seal created and maintained by membrane 250 remains intact until after membrane 250 is punctured by the tip of, e.g., a guide wire, thus helping to facilitate the negative pressure needed to draw blood when aspirating and preventing blood from entering the insertion port during aspiration. In the embodiments depicted in FIGS. 7A and 7B, the perforations or slits on membrane 250 are in the shape of, e.g., a cross (i.e., a “+”). Other shapes of the perforation or slits are contemplated, however, such as those depicted in FIGS. 8, 9, and 10A-10C. For example, membrane 250 may have a single perforation or slit.

Reference is made to FIG. 8, which a top view and side views of a representative membrane 150 according to certain aspects of the present invention. The embodiment of membrane 150 depicted in FIG. 8 comprises a single perforation or slit arranged in its center. During a catheter placement procedure, when the plunger of a syringe is retracted from the syringe's hollow receptacle barrel to test for venous penetration it creates a negative pressure on the downstream side of membrane 150 (i.e., the side of membrane 150 located internally within the connector hub). The negative pressure causes membrane 150 to collapse inward slightly with its edges bucking each other thus maintaining seal, which is made possible by, e.g., the narrowness of the cut and the elastic material of membrane 150.

Reference is made to FIG. 9, which a top view and side views of a representative membrane 152 according to certain aspects of the present invention. The embodiment of membrane 152 depicted in FIG. 9 is essentially the same as the embodiment of membrane 150 depicted in FIG. 8 except that the perforations/slits of membrane 152 are cut on an angle relative to the perforations/slits of membrane 150. Cutting the perforations/slits on an incline or angle as shown in FIG. 9 can further reduce any air leakage while membrane 152 is under a negative pressure caused by withdrawing the syringe plunger while testing for venous penetration.

Reference is made to FIGS. 10A, 10B, and 10C, which are top views of representative membranes 250, 350, and 450 according to certain aspects of the present invention. As depicted in FIGS. 10A, 10B, and 10C, the membrane may comprise differently shaped perforations or slits. The perforations or slits, such as those shown on membranes 150, 152, 250, 350, and 450 depicted in FIGS. 8, 9, 10A, 10B, and 10C can be cut into the thin elastomeric membrane via different techniques such as, e.g., a sharp knife, a steel rule die, a UV or Nd-Yag laser cutting tool, and/or precision micromachining.

The sealing ability of the membranes according to aspects of the present invention can be controlled by the membrane thickness, length of perforations/slits, the type of membrane material, and the membrane's elastomeric properties. Generally, however, membranes according to certain aspects of the present invention are manufactured from polydimethylsiloxane polymer (e.g., polymers in compliance with 21 CFR 177.2600 and USP Class VI) and have a thickness of about 0.030 to about 0.040 inches and a hardness of about 40 durometers. For example, in some embodiments, the thickness of the membrane is about 0.03125 inches, or about 1/32 inches thick.

The membranes according to certain aspects of the present invention may be attached to connector hub 100 in a variety of ways. Generally, in certain embodiments, the circumference of the membrane, such as, e.g., membrane 250, is pressed tightly by a ring-shaped cap that presses against the membrane seat on, e.g., insertion port 140 on connector hub 100. In some embodiments, the ring-shaped cap may be secured to, e.g., first insertion port opening 141 via an adhesive or ultrasonic welding.

Certain embodiments of the present invention include a kit and/or system having a connector hub, such as, e.g., connector hub 100 shown in FIGS. 1, 2A-2B, 3A-3C, 4A-4B, 5A-5C, and 6, along with an introducer needle, such as, e.g., introduction needle 105 shown in, e.g., FIGS. 4A, 4B, and FIG. 6, along with a syringe compatible with the connector hub and introducer syringe. In certain embodiments, an introducer needle system or kit according to certain aspects of the present invention comprises, e.g., four components: an introducer needle; a multi-branched (or multi-port) connector hub; a seal such as, e.g., a membrane or valve; and a syringe. Generally, the introducer needle is formed with a sharp point for puncturing the skin, tissue, and subclavian vein of a patient. In some embodiments, the distal end of the needle is attached and over-molded into the multi-branched (or multi-port) connector hub, which, in certain embodiments, has at least two branches or ports—one branch/port comprising a luer taper assembly configured to engage and connect with a standard syringe, which may function as a handle for the introducer needle system; and another branch/port equipped with a seal (e.g., a membrane or valve) that blocks air penetration but allows easy entry of, e.g., a catheter guide wire without the need for removing the syringe.

Certain embodiments of the present invention also include methods of placing a centerline catheter into the subclavian vein using the described devices of the present invention, including the representative embodiments of connector hub 100 shown in FIGS. 1, 2A-2B, 3A-3C, 4A-4B, 5A-4C, and 6. A method for placing a centerline catheter into the subclavian vein of a patient in need thereof according to certain aspects of the present invention may comprise: providing a connector hub according to certain aspects of the present invention, the connector hub being engaged with an introducer needle at, e.g., a first portion of the connector hub and with a syringe at, e.g., a second portion of the connector hub, and installing a catheter guide wire into the subclavian vein by threading the catheter guide wire through each of an insertion port passage, a medial passage, a first passage, and an internal lumen of the introducer needle.

What is claimed is:

1. A connector hub for a needle assembly for placing a centerline catheter into the subclavian vein of a patient in need thereof, said connector hub comprising:
a first portion located at a first end section of the connector hub, said first portion comprising a first opening, a first passage fluidly connected to said first opening, and a first longitudinal axis;
a second portion located at a second end section of the connector hub opposite said first end, the second portion comprising a second opening, a second passage fluidly connected to said second opening, and a second longitudinal axis;
a medial portion located at a middle section of the connector hub between said first end section and said second end section, said medial portion comprising a medial passage fluidly connected with said first passage and said second passage, and a medial longitudinal axis, wherein said medial longitudinal axis is arranged at an angle relative to said first longitudinal axis of said first portion; and
at least one insertion port located on said medial portion, wherein said insertion port comprises a first insertion port opening, an insertion port passage fluidly connected to said medial passage, and an insertion port longitudinal axis, wherein said first insertion port opening is hermetically sealed, and wherein said insertion port longitudinal axis is arranged at an angle relative to said medial longitudinal axis.

2. The connector hub of claim 1, wherein said first portion is configured to engage a needle having a hollow needle lumen and a needle longitudinal axis, said first longitudinal axis of said first portion being arranged relative to said needle longitudinal axis at an angle of 0 degrees.

3. The connector hub of claim 1, wherein said second portion is configured to engage a syringe having a syringe longitudinal axis, said second longitudinal axis of said second portion being arranged relative to said syringe longitudinal axis at an angle of 0 degrees.

4. The connector hub of claim 1, wherein said medial longitudinal axis is arranged relative to said first longitudinal axis of said first portion at an angle between 20-40 degrees.

5. The connector hub of claim 1, wherein said medial longitudinal axis is arranged relative to said first longitudinal axis of said first portion at an angle of 30 degrees.

6. The connector hub of claim 1, wherein said insertion port longitudinal axis is arranged relative to said medial longitudinal axis at an angle between 20-40 degrees.

7. The connector hub of claim 1, wherein said insertion port longitudinal axis is arranged relative to said medial longitudinal axis at an angle of 30 degrees.

8. The connector hub of claim 1, wherein said insertion port longitudinal axis is arranged relative to said first longitudinal axis at an angle of 20-40 degrees.

9. The connector hub of claim 1, wherein said insertion port longitudinal axis is arranged relative to said first longitudinal axis at an angle of 40 degrees.

10. The connector hub of claim 1, wherein the insertion port passage has a conical shape comprising a first cone diameter and a second cone diameter, wherein said second cone diameter is larger than said first cone diameter, and wherein said first cone diameter is arranged proximal to said medial passage.

11. The connector hub of claim 1, wherein said first insertion port opening is arranged flush with an external surface of said medial portion.

12. The connector hub of claim 1, wherein said first insertion port opening is hermetically sealed with a membrane, wherein a catheter guide wire is configured to puncture said membrane upon inserting said catheter guide wire through said first insertion port opening and into said insertion port passage.

13. The connector hub of claim 1, wherein the connector hub is manufactured from a transparent material that allows for direct visualization of blood as it enters the connector hub.

14. A kit for placing a centerline catheter into the subclavian vein of a patient in need thereof, the kit comprising an introducer needle, a syringe, and the connector hub according to claim 1.

15. A method for placing a centerline catheter into the subclavian vein of a patient in need thereof, the method comprising:
providing the connector hub according to claim 1, said connector hub being engaged with an introducer needle at said first portion and with a syringe at said second portion, the introducer needle comprising a hollow internal lumen;
installing a catheter guide wire into the subclavian vein by threading said catheter guide wire through each of said insertion port passage, said medial passage, said first passage, and said internal lumen of said introducer needle.

16. A method for placing a centerline catheter into the subclavian vein of a patient in need thereof, the method comprising:
providing the connector hub according to claim 1, said connector hub being engaged with an introducer needle at said first portion and with a syringe at said second portion, the introducer needle comprising a hollow internal lumen;
installing a catheter guide wire into the subclavian vein by threading said catheter guide wire through each of said second passage, said medial passage, said first passage, and said internal lumen of said introducer needle.

17. A method of manufacturing the connector hub according to claim 1, wherein the connector hub is manufactured by a method selected from the group consisting of injection molding, 3D printing, extrusion blow molding, injection blow molding, and vacuum casting.

18. A connector hub comprising:
a first portion located at a first end section of the connector hub, said first portion comprising a first opening, a first passage fluidly connected to said first opening, and a first longitudinal axis;
a second portion located at a second end section of the connector hub opposite said first end, the second portion comprising a second opening, a second passage fluidly connected to said second opening, and a second longitudinal axis;
a medial portion located at a middle section of the connector hub between said first end section and said second end section, said medial portion comprising a medial passage fluidly connected with said first passage and said second passage, and a medial longitudinal axis, wherein said medial longitudinal axis is arranged at an angle relative to said second longitudinal axis of said second portion; and
at least one insertion port located on said medial portion, wherein said insertion port comprises a first insertion port opening, an insertion port passage fluidly connected to said medial passage, and an insertion port longitudinal axis, wherein said first insertion port opening is hermetically sealed, and wherein said insertion port longitudinal axis is arranged at an angle relative to said medial longitudinal axis.
